(19) United States
(12) Patent Application Publication
(10) Pub. No.: US 2023/0408501 A1
(43) Pub. Date: Dec. 21, 2023

(54) DETECTION OF TARGET ANALYTES AT PICOMOLAR CONCENTRATIONS

(71) Applicant: The Regents of the University of California, Oakland, CA (US)

(72) Inventors: Ahmet Ali Yanik, Santa Cruz, CA (US); Xiangchao Zhu, Santa Cruz, CA (US)

(73) Assignee: The Regents of the University of California, Oakland, CA (US)

(21) Appl. No.: 18/241,275

(22) Filed: Sep. 1, 2023

Related U.S. Application Data
(63) Continuation of application No. 16/335,176, filed on Mar. 20, 2019.
(60) Provisional application No. 62/400,307, filed on Sep. 27, 2016.

(51) Int. Cl. G01N 33/543; B01L 3/00; B03C 1/01; B03C 1/28; B03C 1/30; G01N 27/48; G01N 27/74

(52) U.S. Cl. CPC G01N 33/54333; B01L 3/502761; B03C 1/01; B03C 1/288; B03C 1/30; B03C 2201/26; G01N 27/745; G01N 33/5438; B01L 2200/0668; B01L 2300/0896; B01L 2400/043; G01N 27/48

(57) ABSTRACT

Methods for detecting submolar concentrations of a target analyte in a sample are disclosed. These methods combine a process of biomarker to bead conversion with bead enrichment and simple visual, optical, or electrochemical detection of the presence of enriched beads to provide sensitive and inexpensive assay for detecting analytes in a sample. Devices for performing these methods are also disclosed.

### CROSS-REFERENCE

This application claims the benefit of U.S. Provisional Patent Application No. 62/400,307, filed Sep. 27, 2016, which application is incorporated herein by reference in its entirety.

### INTRODUCTION

Detection and monitoring of disease progression or recurrence is impeded by a dependence on specialized labs and highly skilled personnel to perform the requisite assays used in a clinical setting for the effective diagnosis and treatment of diseases.

Integration of microfluidic technology, electronics, and nano- and microscale materials can provide real-time monitoring of molecular signatures of disease using small volumes of clinical samples that include proteins, nucleic acids and other chemical species. Quantitative polymerase chain reaction (qPCR) and enzyme-linked immunosorbent assay (ELISA) have proven to be effective non-invasive screening methods for detecting and quantitating nucleic acids and protein targets of interest from clinical samples.

However, these techniques require sophisticated experimental operating procedures, long incubation times, labeling of molecular species, and expensive and bulky instruments. Despite all these, the limit of detection of the target molecules is unacceptably high. As a result, the applicability and adaptability of medical diagnostic systems are restrained—most particularly in resource limited countries. Sensitivity and specificity of detection can be compromised by background noise resulting from factors such as the intrinsic heterogeneity of proteins and other chemical species in complex clinical samples such as blood or urine. Although ex situ sample processing can improve sensitivity and specificity, such processing involves time consuming filtering, centrifugation, and desalting and buffer exchange steps that slow down the turnaround time of diagnosis.

In addition, measurement of biomolecular species at extremely low concentrations can be required to differentiate a disease state from a healthy state and/or to monitor disease progression at earlier stages in the disease process. Typically, the concentration of biomolecular species of interest in an unprocessed biological sample at an early stage of disease ranges from tens of attomolar (˜10⁻¹⁷ M) to picomolar ˜(10⁻⁹ M). In a typical detection system, very few target biomolecular species will diffusively and randomly transit to a sensing surface with a small footprint. Such a system would require an impractically long incubation time (hours to days) to detect such molecular species. Meanwhile, the broad dynamic range of detection diminishes the sensitivity and therefore the reliability of the assay.

Therefore, there is a need to develop devices and methods that provide the effective detection of biomolecular species in clinical samples.

### SUMMARY

A method for detecting presence of a target analyte in a sample is provided. In certain embodiments, the method includes: i) generating a two-particle complex comprising the target analyte sandwiched between a magnetic bead and a dielectric bead; ii) contacting the two-particle complex with a dissociation solution to dissociate the two-particle complex and release dielectric beads present in the two-particle complexes; iii) applying magnetic field to immobilize the magnetic beads present in or released from the two-particle complex; iv) detecting the presence of dielectric beads in the dissociation solution by flowing the dissociation solution through a substrate comprising an array of nanoholes, wherein the diameter of the nanoholes is smaller than the diameter of the dielectric beads, wherein the presence of dielectric beads indicates that the target analyte is present in the sample, and wherein the presence of dielectric beads is detected by: (a) visual observation by a user of presence of the dielectric beads on the array; (b) optical detection using a photodetector; or (c) measuring occlusion of the nanoholes by the dielectric beads as indicated by decrease in an electrical signal from the nanoholes.

In certain embodiments, the two-particle complex comprises a magnetic bead conjugated to a first binding element that specifically binds to the target analyte and a dielectric bead conjugated to a second binding element that specifically binds to the target analyte. In certain embodiments, the first binding element is a first antibody that specifically binds to the target analyte and the second binding element is a second antibody that specifically binds to the target analyte.

In certain embodiments, step (i) includes contacting the sample with magnetic beads comprising a first binding element immobilized on the magnetic beads, wherein the first binding element binds to the target analyte to form a first complex comprising the target analyte bound to the magnetic beads; contacting the first complex with dielectric beads comprising a second binding element immobilized on the dielectric beads, wherein the second binding element binds to the target analyte to form the two-particle complex. In further embodiments, method also includes applying a magnetic field to the two-particle complex thereby immobilizing the two-particle complex and removing dielectric beads not present in the two-particle prior to performing step (ii).

In other embodiments, the two-particle complex comprises a magnetic bead conjugated to a first binding element that specifically binds to the target analyte, a second binding element that specifically binds to the target analyte, and a dielectric bead conjugated to a third binding element that specifically binds to the second binding element. In certain embodiments, the first binding element is a first antibody that specifically binds to the target analyte, the second binding element is a second antibody that specifically binds to the target analyte, and the third binding element is a third antibody that specifically binds to the second antibody.

In another embodiment, the two-particle complex comprises a magnetic bead conjugated to a first binding element that specifically binds to the target analyte, a second binding element that specifically binds to the target analyte, wherein the second binding element is conjugated to a first member of a high-affinity binding couple, and a dielectric bead conjugated to a third binding element which is a second member of the high-affinity binding couple. In certain cases, the first member of the high-affinity binding couple is biotin and the second member of the high-affinity binding couple is avidin or other biotin binding protein. In certain cases, the first member of the high-affinity binding couple is avidin or other biotin binding protein and the second member of the high-affinity binding couple is biotin. In certain cases, step (i) includes contacting the sample with magnetic beads comprising a first binding element immobilized on the magnetic beads, wherein the first binding element specifically binds to the target analyte to form a first complex comprising the target analyte bound to the magnetic beads; contacting the first complex with a second binding element, wherein the second binding element specifically binds to the target analyte to form a second complex comprising the second binding element bound to the target analyte in the first complex; and contacting the second complex with dielectric beads comprising a third binding element immobilized on the dielectric beads, wherein the third binding element specifically binds to the second binding element to form the two-particle complex comprising dielectric beads bound to the second complex. In some cases, the method further includes applying a magnetic field to the two-particle complex thereby immobilizing the two-particle complex; and removing dielectric beads not present in the two-particle complex.

In some cases, step (ii) includes contacting the two-particle complexes with a dissociation solution to dissociate the two-particle complexes and release dielectric beads present in the two-particle complexes while the two-particle complex is suspended in solution or is immobilized by a magnetic field.

In certain embodiments, visual observation by a user of the presence of the dielectric beads on the array comprises seeing the dielectric beads. In certain embodiments, visual observation by a user of the presence of the dielectric beads on the array comprises observing a resonance shift caused by presence of the dielectric beads on the array. In certain embodiments, the array includes a nanoplasmonic surface and wherein the presence of the dielectric; beads on the array surface results in a resonance shift observable by a user.

In certain embodiments, optical detection comprises detection of an optical signature of the dielectric bead by a photodetector. The photodetector may be a fluorescence detector or a spectrophotometer.

In certain embodiments, the detecting comprises measuring occlusion of the nanoholes by the dielectric beads as indicated by decrease in an electrical signal from the nanoholes. In some cases, the array of nanoholes is disposed in an electrochemical cell comprising a first chamber and a second chamber separated by the array and wherein the method comprises: introducing the dissociation solution into the first chamber, flowing the dissociation solution through the array and into the second chamber; and measuring an electrical signal in the second chamber wherein a decrease in the electrical signal over time indicates presence of dielectric beads on the array.

In certain embodiments, the method includes applying a magnetic field to the first complex thereby immobilizing the first complex; and contacting the first complex with a wash solution to remove molecules not bound to the first complex prior to contacting the first complex with (a) the second binding element or (b) the second binding element and the dielectric beads.

In certain embodiments, the method includes removing the magnetic field prior to contacting the first complex with (a) the second binding element or (b) the second binding element and the dielectric beads.

In certain embodiments, the method includes applying magnetic field to the second complex thereby immobilizing the second complex; and contacting the second complex with a wash solution to remove molecules not bound to the second complex prior to contacting the second complex with the dielectric beads. In certain embodiments, the method includes removing the magnetic field prior to contacting the second complex with the dielectric beads.

In certain aspects, step i) comprises contacting the sample with magnetic beads and the first binding element, wherein the magnetic beads and the first binding element are functionalized to enable immobilization of the first binding element on the magnetic beads to provide the magnetic beads comprising the first binding element.

In other aspects, step i) comprises simultaneously contacting the sample with the magnetic beads comprising the first binding element immobilized on the magnetic beads and with the second binding element.

In certain embodiments, the step i) comprises simultaneously contacting the sample with the magnetic beads comprising the first binding element immobilized on the magnetic beads, the second binding element and the dielectric beads comprising the third binding element immobilized on the dielectric beads.

In other cases, the method comprises simultaneously contacting the first complex with the second binding element and the dielectric beads comprising the third binding element immobilized on the dielectric beads.

### BRIEF DESCRIPTION OF THE DRAWINGS

FIG. 1 provides a schematic for a biomarker-to-bead (B2B) conversion process for visual detection of presence of a biomarker and/or quantification of a biomarker.

FIG. 2 provides a schematic of an assay design for bead-to-biomarker conversion.

FIG. 3A shows a substrate comprising an array of nanoholes through which a sample potentially containing dielectric beads is flowed.

FIG. 3B illustrates resonance wavelength shift in response to a sample flowing through the array of nanoholes versus flowing over the array of nanoholes. The shift in resonance is in response to protein accumulation on a sensing surface of the array illustrating that flowing a sample through the array versus over the array improves mass transport.

FIGS. 4A-4C illustrate quantitative measurement of biomarker concentration. FIG. 4A. A graph correlating wavelength to normalized transmission demonstrates the benefits of arraying nanoholes with varying resonance wavelengths on a single microfluidic channel. FIG. 4B. Depending on the array resonance wavelength and resonance wavelength shift, the EOT peak of the one of the nanohole sensors will overlap with the transmission window of the band pass filter. The brightest text will indicate the quantitative measure of the disease biomarker. FIG. 4C. The nanohole pattern can be patterned to transmit light in a text format. SEM images and an optical transmission signal are shown.

FIG. 5A. Schematic of an embodiment of a lateral flow assay device for B2B conversion.

FIG. 5B. Elution solution is added and collected back using a pipette.

FIG. 5C. Elution buffer with DNPs is collected for further analysis.

FIG. 6 illustrates an embodiment of a microfluidic set up for the B2B conversion.

FIG. 7 illustrates a schematic of an embodiment of a B2B conversion system on a lab-chip platform, showing initial steps of the B2B conversion assay.

FIG. 8 illustrates a schematic of an embodiment of B2B conversion system of a lab-chip platform, showing final steps of a B2B conversion assay.

FIGS. 9A-9C illustrate that the mass transport limitation of flow-over fluid movement is overcome by flow-through. FIG. 9A. Fluorescence intensity is significantly increased under flow through versus flow over. FIG. 9B. Fluorescence signal on the surface of the nanohole array when using conventional microfluidics is not easily visible to the naked eye. FIG. 9C. Fluorescence signal due to nanofluidic enrichment by flowing the fluid through the nanohole array is easily visible.

FIG. 10A illustrates the detection of as few as 100 dielectric beads as well as an increase in spectral shift with increasing number of dielectric beads accumulating at the surface of a nanohole array.

FIG. 10B illustrates that dielectric beads corresponding to a biomarker concentration of 1 pM are detectable by naked eye after capture on a nanohole array.

FIGS. 11A-11B show data for resonance shift measurements over time. FIG. 11A. Data is shown for different concentrations of a target analyte in phosphate-buffered saline (PBS). FIG. 11B. Data is shown for different concentrations of target analyte in human serum.

FIGS. 12A-12B show recorded data for resonance shift measurements over time. FIG. 12A. Data for a negative control experiment (0 pM of antigen) is compared to data for a sample (15 pM antigen). FIG. 12B. Data for samples with increasing concentrations of Ebola VP40.

FIG. 13A provides a schematic of an embodiment of a B2B conversion scheme for converting proteins of interest into sub-micron sized dielectric beads and enrichment of these dielectric beads. FIG. 13B. Provides a schematic of an electrochemical cell for quantification of the electrochemical response due to bead accumulation on the surface of nanohole array sensor.

FIG. 14 illustrates an embodiment of a B2B conversion assay.

FIGS. 15A-15B show recorded data obtained using cyclic voltammetry. FIG. 15A. Data showing potential scanned at a specified range. FIG. 15B. A smaller potential window was used to accommodate a smaller range.

FIGS. 16A-16D illustrate aspects of quantification system (post-B2B conversion) in an electrochemical cell. FIG. 16A. A simple circuit equivalent diagram of an electrochemical cell. FIG. 16B. SEM image of dielectric beads on nanohole array. FIG. 16C. Real time current signal change using SWV. FIG. 16D. Real time impedance signal change using EIS.

FIGS. 17A-17B show relative current and impedance changes for different biomarker concentrations compared to negative controls. FIG. 17A. Data for current response to different biomarker concentrations compared to negative control. FIG. 17B. Data for impedance response to different biomarker concentrations compared to negative control.

FIGS. 18A-18B illustrate multiplex analyte detection format and detection of green and red fluorescent dielectric beads which correlate to 10 fM and 1 fM of target antigen-I and target antigen-II in the sample solution.

### DEFINITIONS

“Bead” and “particle” are used herein interchangeably and refer to a substantially spherical solid support.

“Nanoparticle(s)” and “nanobead(s)” are used interchangeably herein and refer to the dielectric beads used in the present methods and devices and are generally beads or particles of less than 1 micron in diameter, e.g., between 25 nm and 900 nm in diameter.

As used herein, a “pore” (alternately referred to herein as “nanopore”) or “channel” (alternately referred to herein as “nanopore” or a “nanochannel”) or nanohole refers to an orifice, gap, conduit, or groove in a substrate, where the hole or pore or channel is of sufficient dimension that allows passage of analyte molecules and other microscopic molecules while preventing passage of the bead/particles.

“Dielectric beads” and “dielectric nanoparticles” are used herein interchangeably and refer to a substantially spherical solid support made of substantially non-conductive and non-magnetic material such that these beads are unresponsive to and do not affect magnetic and/or electric field. The dielectric beads may be substantially opaque, transparent, colored, or fluorescent. In certain embodiments, the dielectric beads are larger than the diameter of the nanoholes such that the dielectric beads cannot traverse through the nanoholes present in the array of nanoholes disclosed herein.

“Magnetic beads” or “magnetic particles” are used herein interchangeably and refer to a substantially spherical solid support made of magnetic or paramagnetic material such that these beads are responsive to a magnetic field.

The term “contacting” means to bring or put together. As such, a first item is contacted with a second item when the two items are brought or put together, e.g., by touching them to each other or combining them in the same solution.

The phrase “optically detectable signature” refers to a light signal that can be detected by a photodetector, e.g., a light microscope, a spectrophotometer, a fluorescent microscope, a fluorescent sample reader, or a florescence activated cell sorter, and etc. “Optically detectable signature” may be made up of one or more signals, where the signal is produced by a label. An optically detectable signature may be made up of: a single signal, a combination of two or more signals, ratio of magnitude of signals, etc. The signal may be visible light of a particular wavelength. An optically detectable signature may be a signal from a fluorescent label(s). For example, the “optically detectable signature” for Cy5 is a visible light at the wavelength of 670 nm.

The phrase “distinguishable labels” or any grammatical equivalent thereof refers to labels can be independently detected and measured, even when the labels are mixed. In other words, the amounts of label present (e.g., the amount of fluorescence) for each of the labels are separately determinable, even when the labels are co-located (e.g., in the same tube or in the same duplex molecule or in the same cell). Suitable distinguishable fluorescent label pairs include Cy-3 and Cy-5 (Amersham Inc., Piscataway, NJ), Quasar 570 and Quasar 670 (Biosearch Technology, Novato CA), Alexafluor555 and Alexafluor647 (Molecular Probes, Eugene, OR), BODIPY V-1002 and BODIPY V1005 (Molecular Probes, Eugene, OR), POPO-3 and TOTO-3 (Molecular Probes, Eugene, OR), and POPRO3 and TOPRO3 (Molecular Probes, Eugene, OR). Further suitable distinguishable detectable labels may be found in Kricka et al. (Ann Clin Biochem. 39:114-29, 2002).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an analyte” includes a plurality of such analytes and reference to “the binding element” includes reference to one or more binding elements and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

Methods for detection of target analytes in a sample are provided. These methods provide detection of submicromolar amounts of target analytes in a sample while utilizing a detection method that can be performed without instrumentation or with minimal instrumentation. The assay methods for detection of a target analyte include a step of capturing the target analyte by using magnetic beads and dielectric beads to generate a two-particle complex; enriching dielectric beads released from the two-particle complex using a nanohole array; and detecting the presence of the dielectric beads on a surface of the nanohole array visually, by using a photodetector, or by using an electrochemical detector. Devices for detecting target analytes using the methods disclosed herein are also disclosed.

### Biomarker-to-Bead Conversion

Aspects of the present methods include correlating the presence of a target analyte to the presence of a dielectric bead at a surface of a nanohole array by capturing the target analyte between a magnetic bead and a dielectric bead to form a two-particle complex, removing any dielectric beads not present in the two-particle complex and dissociating the dielectric beads from the two-particle complex. The dissociated dielectric beads are indicative of presence of the target analyte. The terms “two-particle complex(es)” and “two-bead complex(es)” do not refer to a complex that include only two particles or beads. Rather, the term refers to a complex(es) that includes at least one magnetic particle and one dielectric particle. As would be understood by those of skill in the art, the formation of the two-particle complex may be performed by using any number of formats for forming a sandwich of dielectric and magnetic beads and target analyte. For example, a sample containing or suspected of containing a target analyte may be contacted simultaneously with the magnetic and dielectric beads or sequentially contacted with the magnetic bead followed by the dielectric bead or by the dielectric bead followed by the magnetic bead.

In certain aspects of the disclosed methods, detecting the presence of a target analyte in a sample may include i) generating a two-particle complex comprising the target analyte sandwiched between a magnetic bead and a dielectric bead; ii) contacting the two-particle complex with a dissociation solution to dissociate the two-particle complex and release dielectric beads present in the two-particle complexes; iii) applying a magnetic field to immobilize the magnetic beads present in or released from the two-particle complex; iv) detecting the presence of dielectric beads in the dissociation solution by flowing the dissociation solution through a substrate comprising an array of nanoholes, wherein the diameter of the nanoholes is smaller than the diameter of the dielectric beads, wherein the presence of dielectric beads indicates that the target analyte is present in the sample, and wherein the presence of dielectric beads is detected by: (a) visual observation by a user of presence of the dielectric beads on the array; (b) optical detection by a photodetector; or (c) measuring occlusion of the nanoholes by the dielectric beads as indicated by decrease in an electrical signal from the nanoholes.

In certain aspects of the disclosed methods, detecting the presence of a target analyte in a sample may include a step of (1) contacting the sample with magnetic beads comprising a first binding element immobilized on the magnetic beads, wherein the first binding element binds to the target analyte to form a first complex comprising the target analyte bound to the magnetic beads.

In certain embodiments, the step (1) may include contacting the sample with magnetic beads and the first binding element, where the magnetic beads and the first binding element are functionalized to enable immobilization of the first binding element on the magnetic beads to provide the magnetic beads comprising the first binding element. In other words, the magnetic beads on which the first binding element is immobilized may not be previously prepared and can be formed during step (1). In other embodiments, the magnetic beads may have been generated previously by suitable methods for attaching a first binding element.

In some instances, step (1) may be performed in solution where the magnetic beads are in a suspension. In these instances, the method may optionally include a step of applying a magnetic field to the solution to immobilize the magnetic beads, including any first complexes formed by capture of the target analyte on the magnetic beads and flowing away the sample. The magnetic field may then be removed and the magnetic beads re-suspended in a wash solution to dissociate any molecule bound non-specifically to the magnetic beads, followed by applying magnetic field to recapture the magnetic beads and removing the wash solution.

In certain embodiments, the method further includes a step of (2) contacting the first complex with a second binding element, where the second binding element binds to the target analyte to form a second complex comprising the second binding element bound to the target analyte in the first complex.

In certain embodiments, the first complex (which comprises the magnetic beads on which the target analyte has been captured) may be contacted with the second binding element while the first complex is in solution (in absence of an applied magnetic field) or when the first complex is immobilized by application of a magnetic field. For example, the wash solution may be replaced with a solution comprising the second binding element or the second binding element may be added to a solution comprising the first complex in a buffer (e.g., a wash solution). In embodiments, where the first complex is immobilized by application of a magnetic field and is contacted by the second binding element, the method may include removing the magnetic field to release the first complex (and any magnetic beads not bound to the target analyte/second binding element) into a suspension to allow incubation of the first complex with the second binding element in solution, followed by capture of magnetic beads (which may include magnetic beads not bound to the target analyte/second binding element, first complex and second complex) by applying a magnetic field. After step (2), the method may further include removing any molecules not bound to the magnetic beads by for example, applying a magnetic field to capture all magnetic beads, applying a wash solution, and optionally, removing the magnetic field to resuspend the magnetic beads.

In some embodiments, the method further includes a step of (3) contacting the second complex with dielectric beads comprising a third binding element immobilized on the dielectric beads, wherein the third binding element binds to the second binding element to form a two-particle complex comprising dielectric beads bound to the second complex. While steps (2) and (3) are explained separately, these steps need not be separate or sequential. In some embodiments, steps (2) and (3) may be combined such that the first complex is contact simultaneously with the second binding element and the dielectric beads such that the second binding element can bind to the dielectric beads and then to the first complex to form the two-particle complex.

In some embodiments, the method further includes a step of (4) applying magnetic field to the two-particle complex to immobilize the two-particle complex; followed by a step of (5) removing dielectric beads not bound to the two-particle complex. The step of (5) removing dielectric beads not bound to the two-particle complex may include contacting the two-particle complex immobilized by magnetic field with a wash solution and removing the wash solution. In some embodiments, the method may also include resuspending the two-particle complex in a solution (e.g. wash solution) by removing the magnetic field and recapturing the two-particle complex prior to removing the wash solution.

The method may further include a step of (4) contacting the two-particle complex immobilized by the magnetic field with a dissociation solution to release dielectric beads present in the two-particle complex. The dissociation solution comprising the dielectric beads dissociated from the two-particle complex may then be flowed across an array comprising a plurality of nanoholes or nanoapertures that are sized to be smaller than the size of the dielectric beads. This step results in enrichment of the dielectric beads at a surface of the array which facilitates detection of the presence of even very low concentrations (e.g. 10⁻⁶, 10⁻⁹, 10⁻¹², 10⁻¹⁵, 10⁻¹⁷, 10⁻¹⁸, 10⁻¹⁹, 10⁻²⁰ molar, or lower concentrations) of target analyte in a sample.

The presence of the dielectric beads that have been dissociated from the two-particle complex may be detected by visual observation of the dielectric beads trapped on a surface of the array. An unaided human eye may be able to inspect the array to check for trapped dielectric beads, wherein the presence of dielectric beads is indicative of presence of the target analyte in the sample. In such an embodiment, the dielectric beads may be sized to be visible to naked human eye. Alternatively, a user may be able to use glasses (including reading glasses), a magnifying glass, microscope, or equivalent device to observe the presence of the dielectric beads. In some embodiments, the dielectric beads may be provided in a color that enhances their ease of detection by an aided or unaided human eye.

In some embodiments, instead of or in addition to visual detection of the dielectric beads, the presence of the target analyte in the sample may be indicated by a resonance shift caused by the presence of the dielectric beads on the surface of the array. In certain embodiments, the array of nanoholes may include a nanoplasmonic surface and the presence of the dielectric beads on the array surface results in a resonance shift observable by a user.

In some embodiments, instead of or in addition to visual detection of presence of the dielectric beads, the method may include measuring occlusion of the nanoholes by the dielectric beads as indicated by decrease in an electrical signal from the nanoholes. In certain embodiments, the array of nanoholes may be disposed in an electrochemical cell comprising a first chamber and a second chamber separated by the array, wherein the method comprises introducing the dissociation solution into the first chamber, flowing the dissociation solution through the array and into the second chamber; and then measuring an electrical signal in the second chamber. A decrease in the electrical signal over time indicates the presence of dielectric beads on the array.

In certain embodiments, at least the step of contacting the magnetic beads with the sample; the step of contacting the first complex with the second binding element; and the step of contacting the second complex with the dielectric beads may be carried out in a solution phase in order to optimize diffusion and mass transport of molecules that in turn can increase the chances of the molecules coming in sufficiently close proximity for binding to occur. Thus, the methods disclosed herein are advantageous over other methods, such as, sandwich ELISA that requires attachment of the complex to a surface of the reaction vessel and hence reduced diffusion, resulting in a lower probability of molecules coming in sufficiently close proximity for binding to occur. The present methods thus require shorter incubation time for binding to occur. Hence, incubation times are generally less than 60 mins in total, for example, advantageously less than 60 mins, or less than 30 mins, or less than 20 mins or less than 10 mins, which is much shorter than those of microtiter plates, for example, under the same conditions, a typical microtiter plate assay will take about three hours while the presently disclosed methods using beads will take an hour or less, 10 min-45 min, 10 min-30 min, or 10 min-15 min.

The rate of flow through the array of nanoapertures may be controlled via a pressure difference and may range from 1-100 μl/min. e.g., 1-75 μl/min. 1-50 μl/min. 1-25 μl/min, 2-20 μl/min, op 2-10 μl/min. A final elution buffer (e.g., about 100 μl) is processed within 5-120 mins, for example within 5, 7, 10, or 15, 20, 30 or 60, 90 or 120 or 240 mins.

Biomarker-to-bead conversion allows the efficient capture of a target surrogate—that is, the dielectric beads—which due to their size are captured and concentrated at the nanohole. Unbound target molecules can pass through the nanohole. Advantageously the processing time is less than 120 mins and may be less than 30, 45, 60, 120 mins, or less than 15 mins or less than 10 mins or less than 5 mins. The bio-sensing nanohole surfaces need not be functionalized with specific antibodies, since specificity results from the solution chemistry used in B2B conversion, such that the only thing being detected is the quantity of dielectric beads, which in turn represents of the amount of target bound in the B2B conversion. This method simplifies the manufacturing and integration of microfluidics devices for carrying out the detection of the target analyte and reduces the test time relative to other currently available methods. The specificity of the assay for different biomarkers is a result of the functionalized beads (such as antibody functionalized beads). Hence, by changing the binding element on the beads one can adapt these detection techniques to different target analytes. This allows a generic lab-on-chip platform that can be applied to effectively any biomarker that can be detected by specific binding of a binding element.

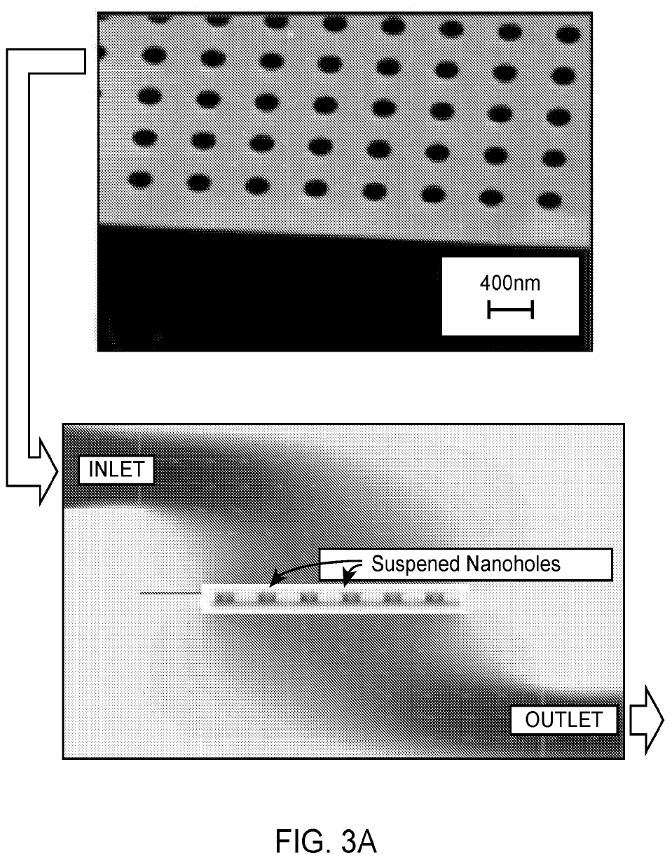

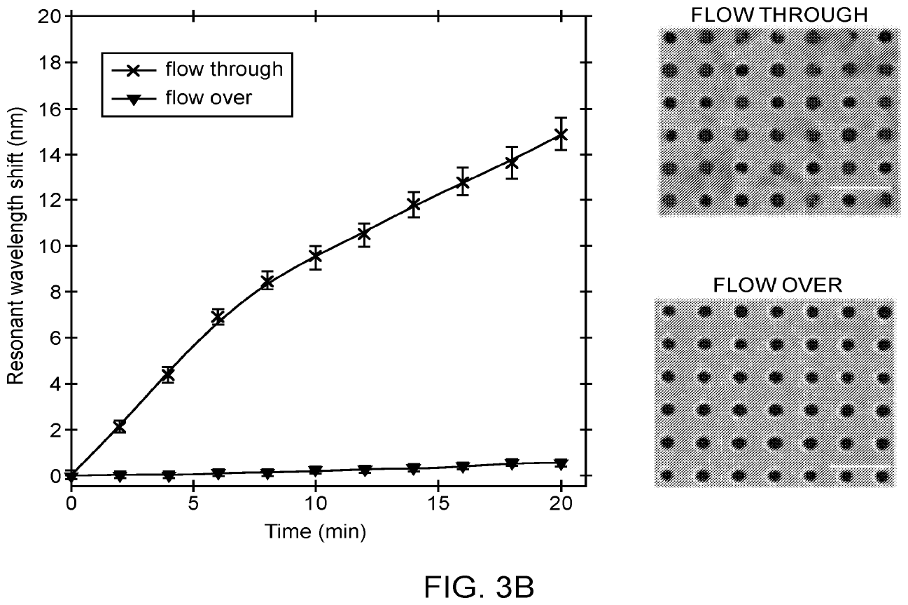

The flow-through approach minimizes the potential loss of dielectric-beads due to non-specific absorption of the beads on channel walls. Dielectric beads have much smaller diffusion constants than biomolecules and tend to follow fluidic streamlines. Hence, a convective flow can bring the dielectric beads to the sensing surfaces more rapidly than with currently available methods that rely on diffusion. As shown in FIG. 3A (with COMSOL simulations), fluidic streamlines go directly towards the array surface and limit dielectric bead interactions with other surfaces. Beads are enriched at the top surface of the array, which is relatively much smaller than the surface of the microfluidic chamber. Hence, the accumulated bead density per unit area is also enriched.

As noted herein, the disclosed methods provide a low-cost assay for the detection of a target analyte. Such detection may be important in field settings where more complex instrumentation and trained technicians are unavailable. For example, these methods can be used to confirm outbreak of a pathogen infection in real time without the delay involved with transporting samples to a laboratory for testing. In some embodiments, the high sensitivity of the present methods (detection of target analytes present at a concentration as low as 10 aM) may be used to detect pathogen infection before the symptoms are apparent. Early detection can be valuable in providing early treatment as well as preventing spreading of the infection. The visual and electrochemical methods implementable in a low cost manner in a filed setting are further described. However, it is noted that these methods are also suitable for a laboratory setting.

### Visual Detection of a Target Analyte

Direct Dielectric Bead Visualization

Direct visual detection of the presence or absence of dielectric beads trapped on a top surface (the surface at which the dissociation solution enters the nanoholes) may be performed by a user by examination of the top surface of the nanohole array. As noted herein, the nanohole array may be contained in a transparent housing or may be contained in a housing with an open top to facilitate observation of the top surface of the nanohole array.

In certain embodiments, the dielectric beads may be colored to facilitate visual observation.

In certain cases, a user may utilize a non-powered magnifying device such as a magnifying glass (e.g., a loupe) in order to observe the beads.

Resonance Shift

In certain embodiments, a resonance shift observable by a human eye may be used to detect presence of dielectric beads trapped on the array of nanoapertures. In certain embodiments, the array of nanoapertures includes a metal film that is opaque and the size of the nanoapertures in the subwavelength range to prevent transmission of light through the array. The incident light can only be transmitted at specific resonant wavelengths (Extraordinary Light Transmission—EOT) through an optical process incorporating surface plasmon polaritons (SPPs).

In certain embodiments, the dissociation solution containing dielectric beads that have been dissociated from the two bead complex may be transported across an array of nanoapertures that includes an opaque metal film and where the size of the nanoapertures is in the subwavelength range to prevent transmission of light through the array. Quantitative measurement of dielectric bead accumulation on the top surface of the array may be performed by spectral analysis of transmission signal, e.g., EOT signal. A spectral shift of 10 nm or more may be indicative of presence of the dielectric beads corresponding to the presence analyte at a particular concentration (e.g. 10 pM).

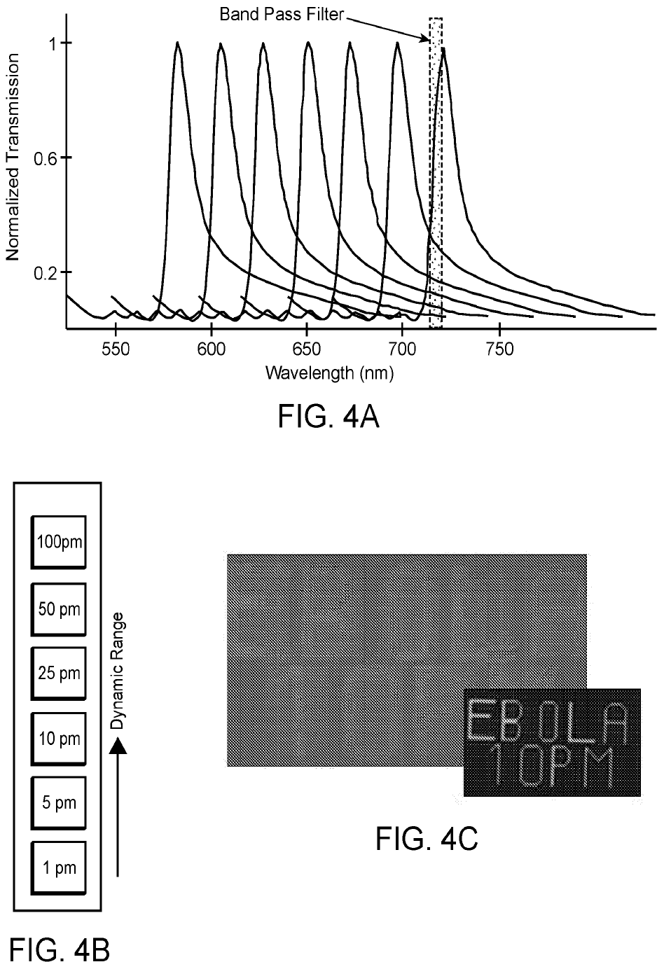

In certain embodiments, quantitative measurement of target analyte concentration may be achieved by arraying a plurality of nanohole arrays in a single channel (e.g., a single microfluidic channel) where different arrays have different resonance wavelengths. In such an embodiment, the resonance shift will depend on the target analyte concentration. Once the resonance wavelength shift causes one of the resonances transmitted by the array to overlap with the transmission window of the band-pass filter (depicted by a rectangular box “Band pass filter” in FIG. 4A), the light intensity from the corresponding array will be maximal. In certain embodiments, the resonance shift may be estimated by arranging the arrays of nanoapertures with varying spectral profiles. In certain cases, the nanohole aperture arrays may be arranged to have a varying spectral profile as illustrated in FIG. 4B. In certain embodiments, the concentration of the target analyte may be encoded to the transmitted light profile by arranging the nanoholes in an array in a text format. In these embodiments, a user can read the measured concentration of the target analyte as illustrated in FIG. 4C. The spectral behavior of the array of nanoapertures may be calibrated to indicate text corresponding to the measured concentration of the target analyte. In certain aspects, a magnifier may be integrated on top surface of the array to facilitate user to read the text.

FIG. 4A illustrates that by arraying nanoholes with varying periodicities on a single microfluidic channel one can cover a larger spectral resonance shift window. FIG. 4B depicts that depending on the array resonance wavelength and resonance wavelength shift, the EOT peak of the one of the nanohole sensors will overlap with the transmission window of the band pass filter. The brightest text will indicate the quantitative measure of the disease biomarker. FIG. 4C illustrates that nanohole array can be patterned to transmit light in a text format. SEM images and optical transmission signal is shown. For example, the type of the disease biomarker and the concentration can be written as in Ebola 10 pM by arraying the nanohole in the text format.

### Optical Detection of Target Analyte Using a Photodetector

In certain cases, detecting the presence or absence of the dielectric beads that have been enriched by trapping them on a surface of the array of nanoholes may be performed using a photodetector. Such an embodiment can be used for detection of a single type of bead having an optically detectable signature or a plurality of dielectric beads, where the dielectric beads have a distinct optically detectable signature.

In certain embodiments, the detection methods disclosed herein may be used to detect presence of two or more different target analytes in a sample by performing the formation of the two-particle complexes in a multiplex format by using appropriately functionalized magnetic beads and dielectric beads and assigning a different optically detectable signature to each set of differently functionalized dielectric beads. For example, a dielectric bead that is functionalized (e.g., by conjugation to a second binding element that binds to a first target analyte) to bind to a first target analyte may be assigned a first optically detectable signature while a dielectric bead that is functionalized (e.g., by conjugation to a second binding element that binds to a second target analyte) to bind to a second target analyte may be assigned a second optically detectable signature.

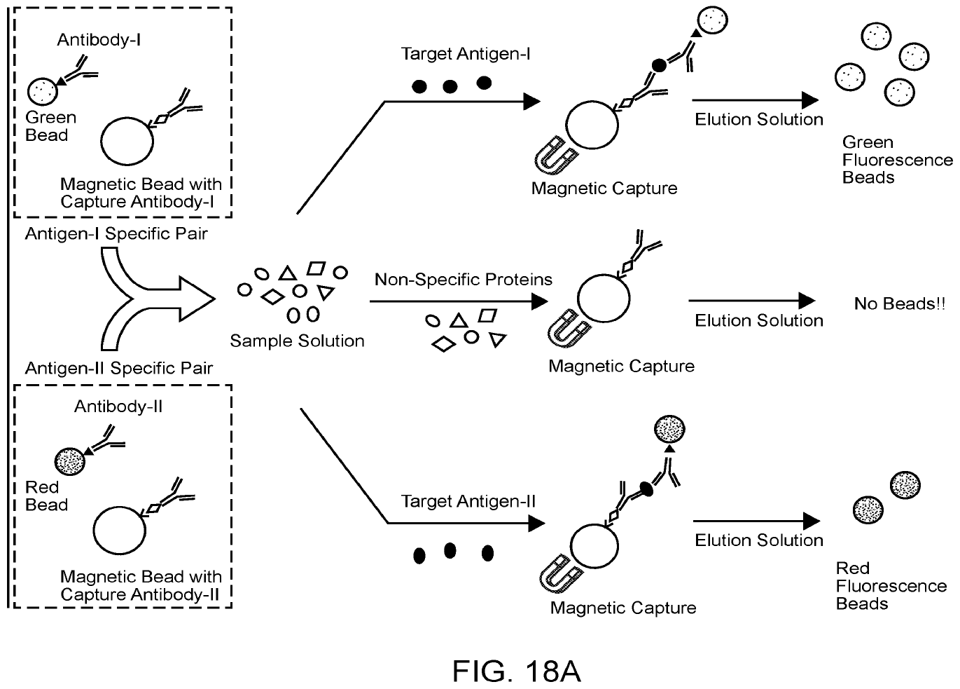

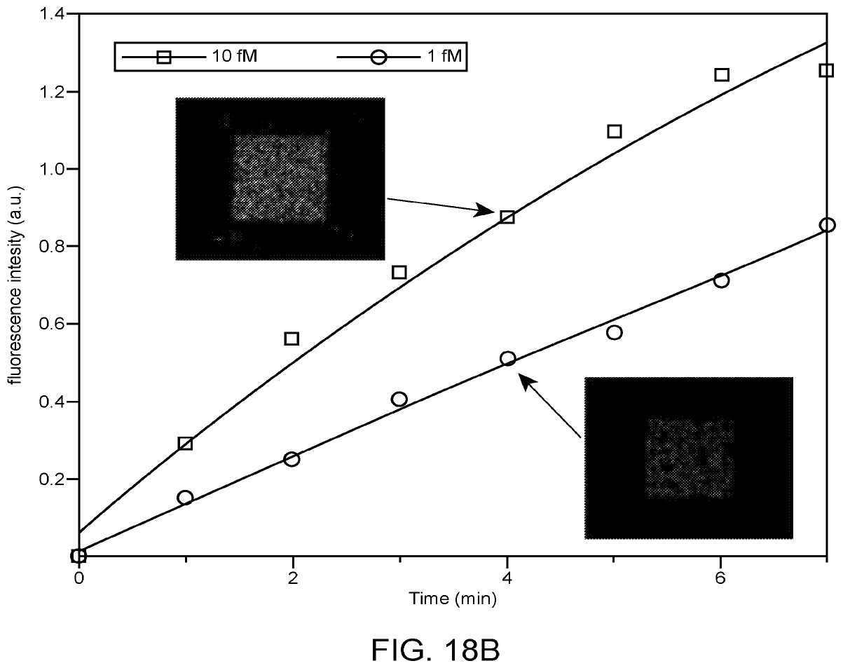

Different optically detectable signatures may be assigned to different dielectric beads by attaching distinguishable labels to the dielectric beads. For example, the dielectric beads may be labeled with different fluorescent labels. FIGS. 18A-18B illustrate multiplex analyte detection format and detection of green and red fluorescent dielectric beads which correlate to 10 fM and 1 fM of target antigen-I and target antigen-II in the sample solution.

As illustrated in FIG. 18A, a sample that included 10 fM of target antigen-I and 1 fM of target antigen-II was contacted with (i) magnetic beads functionalized with capture antibody-I which binds to the target antigen-I and dielectric beads labeled with green fluorescent label and functionalized with antibody-I that binds to target antigen-I; and (ii) magnetic beads functionalized with capture antibody-II which binds to the target antigen-II and dielectric beads labeled with red fluorescent label and functionalized with antibody-II that binds to target antigen-II. Two-particle complexes comprising dielectric beads labeled with green fluorescent label or dielectric beads labeled with red fluorescent label were generated and the dielectric beads eluted from the complexes by contacting the complexes with an elution solution. The elution solution was passed through an array of nanoapertures that enrich the dielectric beads on a top surface by trapping the dielectric beads while the elution solution flows through the nanoapertures in the array. The number of enriched dielectric beads enriched over a period of 6 minutes was proportional to the concentration of the target antigen as seen by the fluorescence intensity of the dielectric beads from the green fluorescence channel (corresponding to 10 fM antigen-I) and from the red fluorescence channel (corresponding to 1 fM antigen-II) (see FIG. 18B). FIG. 18B also includes an image of the top surface of the array of nanoapertures where the dielectric beads were enriched at 4 min after starting collection of dielectric beads at the top surface of the array: the image in the top left corner shows that the dielectric beads labeled with green fluorescent label used to detect the presence of 10 fM target antigen-I are present in greater density than the dielectric beads labeled with red fluorescent label used to detect the presence of 1 fM target antigen-II (image on bottom right corner of FIG. 18B).

### Electrochemical Detection of Target Analyte

In certain embodiments, detecting the presence or absence of the dielectric beads may be performed by detecting a decrease in an electrical signal across the array of nanoholes. In certain embodiments, the dissociation solution (also referred herein as “elution solution”) containing any dielectric beads dissociated from the complex comprising magnetic beads bound to target analytes, may be transferred to a two- or three-electrode electrochemical nanosensor which includes a plurality (e.g., 10,000-10⁶) of perforated nanometer sized fluidic channels (nanohole array) that allow flow-through of ions in the solution including cations (such as Na⁺ or K⁺) and anions (such as PO4⁻ or Cl⁻) to direct the flow of current at the surface of the nanosensor. The dielectric beads are larger than the nanoholes and are prevented from traversing the nanoholes and instead occlude the nanoholes, resulting in a decrease in the flow of ions and a corresponding decrease in current. This amount of signal drop is positively correlated to the number of occluded fluidic channels, which is positively correlated to the concentration of the target analyte.

In certain aspects, the nanohole array may be coated with a conductive material (e.g., Au, Ag, Pt, etc.) that may serve as a working electrode in the electrochemical cell.

In certain aspects, a direct and simple electronic readout (e.g., current vs potential (i-V) response of the nanohole array) indicates the presence or absence of the target analyte.

In another aspect, in addition to detecting the presence of the target analyte, an electrical signal from the nanohole array may be used to quantitate the target analyte. For example, impedance of the bulk aqueous solution flowing through the nanohole array can be used to quantitate the target analyte since the current essentially is governed by the solution resistance. In certain aspects, linear potential sweep voltammetry (square wave voltammetry (SWV)) and impedance spectroscopy (electrochemical impedance spectroscopy (EIS)) may be employed for the determination of the concentration of target analyte.

As noted herein, the nanohole array may be manufactured using a nanofabrication based process, such as, deep ultraviolet lithography, nanoimprinting, nanosphere lithography, nanostencil lithography, etc. In certain aspects, the electrochemical cell may include a first chamber and a second chamber separated by a nanohole array. The electrochemical cell may be housed in a non-conductive material, such as, acrylic or glass. The nanohole array may include a coating of a conductive material to serve as an electrode (e.g., a working electrode) and may be present facing the first chamber. A counter/reference electrode or a counter electrode and a reference electrode may be placed in the second chamber to detect current across the nanohole array. In other embodiments, a working electrode may be disposed in a first chamber and a counter/reference electrode or a counter electrode and a reference electrode may be placed in the second chamber and the nanohole array may not include a layer of conductive material. The working electrode can be the same size as the nanohole array and the counter/reference electrode or a counter electrode and a reference electrode may be 1 cm long or larger. The electrochemical cell may contain a simple salt solution (e.g., NaCl or KCl) to facilitate measurement of electrical signal across the nanohole array.

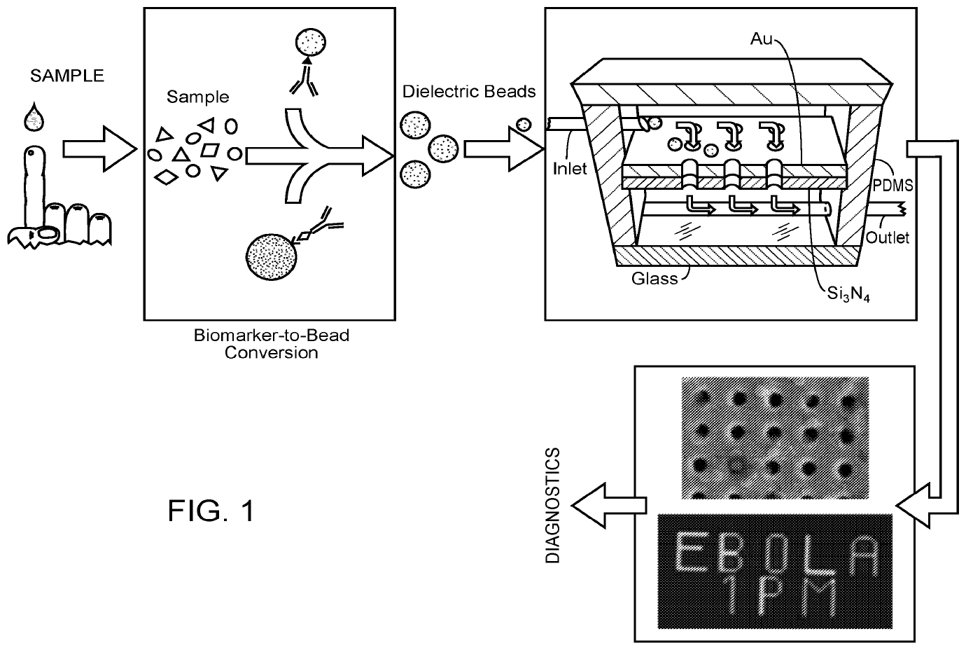

FIG. 1 illustrates an embodiment of a biomarker to bead conversion method disclosed herein followed by enrichment for the dielectric beads that were bound to the target analyte bound to the magnetic beads. The dielectric beads are then dissociated from the complex. The solution containing the dissociated dielectric beads flows through the nanoapertures of an array. The array may be housed in a device comprising a chamber divided by the array into a first chamber and a second chamber. The first chamber may include an inlet for introducing the dissociated dielectric beads into the first chamber and the second chamber may include an outlet for removing the dissociation solution. The first chamber may include an open top to facilitate visualization of any dielectric beads captured on the surface of the array. Alternatively the top of the first chamber or at least one wall of the first chamber may be substantially transparent to facilitate visualization of the surface of the array. The surface of the array on which the dielectric beads are trapped may be referred to as the top surface. The approach outlined in FIG. 1 combines biomarker-to-bead conversion, surface enrichment and naked eye detection to achieve visual detection limits as low as 1 pM. The total assay time is estimated to be <30 mins.

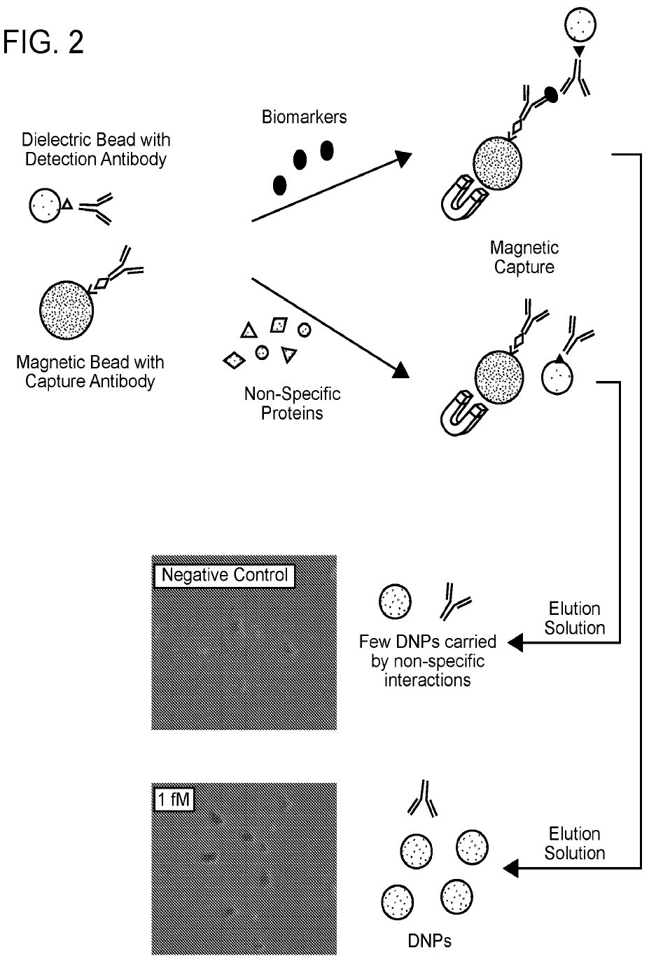

FIG. 2 provides a schematic of B2B conversion protocol followed by enrichment of dielectric beads and visualization of 1 fM of target analyte.

The step of flowing the dissociation solution through a nanopore array to enrich the dielectric beads that correspond to the presence of a target analyte in a test sample enhances detection of the analyte as compared to detection methods that utilize surface capture of target analytes and detection of the captured analyte. For example, in vitro diagnostics (IVD) technologies exploit highly specific biomolecular recognition processes for surface capturing of targets (biomarker proteins, pathogens, tumor cells, etc.). However, detection limits are often hindered by the inefficient delivery of the targets by random diffusion, to the sensing surfaces. Hence, instead of counting on the randomized diffusive transport of the targets, the disclosed methods divert convective flow (fluidic streamlines) directly towards the array and enhance the exponential mass transport constant efficiency by at least 2-fold, 3-fold, 5-fold, 7-fold, 14-fold, or at least 20-fold, or an exponential increase in mass transport efficiency.

To visually verify the presence or absence of dielectric beads (representing, for example, the presence or absence of biomarker protein or nucleic acid in the original sample) in the elution solution, the solution is routed through the suspended nanoholes (a nanohole array, for example in a membrane) and dielectric beads are accumulated (enriched) at the nanoholes. Typical flow rates vary from 10-100 μl/min, for example, μl/min, 10-50 μl/min, 10-30 μl/min, 10-20 μl/min, or at least 10 μl/min, at least 15 μl/min or at least 20, 50 or 100 or 200 μl/min. A final elution buffer (e.g., about 100 μl) is processed (e.g., transferred across the nonohole array) within 1-120 mins, for example within 1-60 min, 1-30 min, 5-20 min, 5-10 min, or within 5, 7, 10, or 15, 20, 30 or 60, 90 or 120 or 240 mins. In certain embodiments, the total time between starting the assay and obtaining a result indicative of presence or absence of the target analyte may be less than 30 minutes.

In certain embodiments, the method for detecting the presence of an analyte in a sample may include (i) Contacting, in solution, functionalized magnetic beads with the sample putatively containing a target analyte, the functionalized magnetic beads comprising a first binding element (e.g., a first antibody) that binds to the target analyte; (ii) Contacting a second binding element with the solution containing the functionalized magnetic beads and the sample putatively containing a target compound. The second binding element has a first binding portion (e.g. the Fc portion of an antibody) that is capable of binding to a functionalized dielectric bead, and a second binding portion (e.g. the Fab portion of an antibody) that binds specifically to the target analyte. (iii) Applying a magnetic field to attract and isolate the functionalized magnetic beads together with all bound elements, attracting them to and concentrating them on a surface (for example the inside of a container, e.g. a tube). (iv) Replacing the solution with a wash solution and removing the wash solution to remove the elements not immobilized by the magnetic field, leaving the functionalized magnetic beads together with all attached elements. (v) Releasing the magnetic field and re-suspending the functionalized magnetic beads in a first volume or a first solution. This is called “the resuspension solution”. (vi) Adding to the resuspension solution a plurality of dielectric beads coated with part two of a two-part high-affinity binding couple, for example biotin (or biotin related compound or Avidin/streptavidin/etc., provided it performs the function of binding with high affinity to the part one of the two-part high-affinity binding couple. (vii) For a second time, applying a magnetic field to attract and isolate the functionalized magnetic beads together with all bound elements, attracting them to and concentrating them on the surface. (viii) While the magnetic field is still maintained, washing the magnetically bound elements to remove the elements not bound by the magnetic field (e.g., unbound dielectric beads), leaving the functionalized magnetic beads together with all attached elements including the dielectric beads which are attached to the magnetic beads if the target was present in the sample. (ix) While the magnetic field is still maintained, contact (rinse) the magnetically bound elements with a dissociation solution (elution solution) that reverses the interaction between at least one of the first, second, or third binding elements, e.g., neutralizes or denatures the target proteins or other proteins that maintain the integrity of the two bead complex, thereby releasing the dielectric beads from the magnetic beads into solution, and producing a solution (the assay solution) containing the dielectric beads only (if any). (x) Enrich and accumulate the dielectric beads at a surface of the nanoaperture array by passing the solution (the ‘assay solution’) containing the eluted dielectric beads through the array whereby dielectric beads are accumulated (enriched) at the top surface (at which the solution enters the naoapertures) of the array. (xi) Detecting the accumulated dielectric beads by:
(a) visual observation of the dielectric beads or resonance shift of light incident on the top surface of the array; or
(b) detecting conductance through the nanoapertures.

The dielectric beads may be detected by the naked eye or by using surface plasmon resonance to produce a signal at the top surface, wherein the signal is visible with the naked eye without the use of powered magnification.

In other embodiments detection is done using non-powered magnification, for example using a lens, magnifying glass, loupe or microscope, but without the use of any electrically powered magnification or image intensifying device. Other embodiments may use electrically powered magnification.

Quantification or semi-quantification of the target analyte may be achieved in numerous standard ways such as by measuring or observing or recording the number of dielectric beads accumulated on the array.

The steps of contacting the sample with the beads and/or binding elements may be performed under suitable conditions and time to enable interactions leading to formation of two-bead complexes (magnetic beads bound to dielectric beads via target analyte). In certain cases, the contacting step may be an incubation step in the presence of a buffer (e.g., a buffer providing a physiological environment). The contacting may be performed at room temperature, at 37° C., at 4° C., or any suitable temperature.

The elution buffer (also referred to as dissociation solution) may include agents that reverse one or more interactions holding the two-bead complex together. In certain embodiments, the elution buffer may be a high salt buffer or may include chaotropic agents that disrupt protein-protein interactions.

As used herein, the terms “sample”, “test sample”, “biological sample” refer to fluid sample containing or suspected of containing an analyte of interest. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing the analyte may be assayed directly. The source of the analyte molecule may be synthetic (e.g., produced in a laboratory), the environment (e.g., air, soil, etc.), an animal, e.g., a mammal, a plant, or any combination thereof. In a particular example, the source of an analyte is a human bodily substance (e.g., blood, serum, plasma, urine, saliva, sweat, sputum, semen, mucus, lacrimal fluid, lymph fluid, amniotic fluid, lung lavage, cerebrospinal fluid, feces, tissue, organ, or the like). Tissues may include, but are not limited to skeletal muscle tissue, liver tissue, lung tissue, kidney tissue, myocardial tissue, brain tissue, etc. The sample may be a liquid sample or a liquid extract of a solid sample. In certain cases, the source of the sample may be an organ or tissue, such as a biopsy sample, which may be solubilized by tissue disintegration/cell lysis. A sample may be processed prior to performing the disclosed detection methods on the sample. For example, the sample may be concentrated, diluted, purified, amplified, etc.

The methods and devices disclosed herein may be used to detect a target analyte in sample volumes as small as 200 μl or less, 100 μl or less, or 50 μl or less, for example, less than 30 μl, 10 μl, 5 μl, 1 μl, or smaller. The methods and devices disclosed herein may be used to detect a target analyte present in a sample at a concentration lower than 1 μM, e.g., lower than 10 nM, 1 nM, 10 pM, 1 pM, 10 aM, 1 aM, 10 fM, or 1 fM. The methods and devices disclosed herein may be used to detect a target analyte present in a sample at a concentration of 10 pM-1 fM, e.g., 1 pM-10 fM, 100 aM-100 fM, 30 aM-100 fM, or 10 aM-100 fM.

As will be appreciated by those in the art, the first and second binding elements will be selected based on their ability to bind to the target analyte. Binding elements for a wide variety of target molecules are known or can be readily found or developed using known techniques. For example, when the target analyte is a protein, the binding members may include proteins, particularly antibodies or fragments thereof (e.g., antigen-binding fragments (Fabs), Fab′ fragments, F(ab′)₂ fragments, full-length polyclonal or monoclonal antibodies, antibody-like fragments, etc.), other proteins, such as receptor proteins, Protein A, Protein C, or the like.

In the case where the analyte is a small molecule, such as, steroids, bilins, retinoids, and lipids, the first and/or the second binding element may be a scaffold protein (e.g., lipocalins) or an aptamer. In some cases, the first and second binding elements for protein analytes may each be a peptide. For example, when the target analyte is an enzyme, suitable binding elements may include enzyme substrates and/or enzyme inhibitors which may be a peptide, a small molecule or other enzymatic substrate, derivative, or mimic thereof. In some cases, when the target analyte is a phosphorylated species, the binding elements may comprise a phosphate-binding agent. In certain cases, the first and second binding elements may be aptamer, a polynucleotide (also referred to as a nucleic acid), such as DNA, RNA, (including oligonucleotides or modified oligonucleotides thereof), and the like. In certain embodiments, the first binding element may be a first antibody or an antigen binding fragment thereof that binds to a first epitope of the target analyte and the second binding element may be a second antibody or an antigen binding fragment thereof that binds to a second epitope of the target analyte. In another embodiment, the target analyte may be a nucleic acid (e.g., DNA or RNA) and the first binding element may be a nucleic acid that is complementary to a first sequence present in the target nucleic acid and the second binding element may be a nucleic acid that is complementary to a second sequence present in the target nucleic acid. In another embodiment, the target analyte may be a peptide and the first binding element may be an enzyme that binds to the peptide and the second binding element may be an antibody or aptamer that specifically binds to the peptide. Any suitable combination of first and second binding elements may be used provided they can simultaneously bind to the target analyte.

In some embodiments, a third binding element is used. The third binding element may be any molecule that binds to the second binding element provided that the binding of the third binding element does not interfere with the binding of the second binding element to the target analyte. In some embodiments, the third and second binding elements may be ‘a two-part high-affinity binding couple’ where third and second binding elements bind to one another with strong binding kinetics, such as the Avidin-biotin couple, including the moieties Avidin, NeutrAvidin, or streptavidin or biotin or an Avidin or biotin related compound. For example, the second binding element may be an antibody that binds to the target analyte and may be conjugated to a first member of a binding pair, e.g., a first member of a two-part high-affinity binding couple and the third binding element may be a second member of a binding pair, e.g., a second member of a two-part high-affinity binding couple. In some cases, the second binding element may be a first antibody and the third binding element may be a second antibody that binds to the first antibody (e.g., to the Fc region of the antibody). In a particular example, the first binding element may be a first antibody, e.g., an IgG antibody that binds to a first epitope on a target analyte; the second binding element may be a second antibody (e.g., IgM) that binds to a second epitope on the target analyte, wherein the second binding element is functionalized with conjugation to a biotin molecule; and the third binding component may be an avidin molecule disposed on dielectric beads.

The terms “target analyte,” “target molecule,” “biomarker,” and “analyte or “molecule of interest” are used interchangeably to refer to a molecule that is being detected in a test sample. An analyte may be a small molecule, peptide, protein, RNA, DNA, lipid, carbohydrate, toxin, or a cell. In certain embodiments, the target analyte may be a biomarker for a pathogen, such as, a virus or a bacteria. In certain embodiments, the target analyte may be a protein or a nucleic acid from a pathogen, such as, Ebola virus (EBOV) protein or nucleic acid, HIV protein or nucleic acid, and the like. In other embodiments, the target analyte may be a protein or nucleic acid associated with cancer such as a cancer antigen.

In certain embodiments, the first and second binding elements bind specifically to the analyte. By “specifically bind” or “binding specificity,” it is meant that the binding element binds the analyte molecule with specificity sufficient to differentiate between the analyte molecule and other components or contaminants of the test sample. For example, the binding element, according to one embodiment, may be an antibody that binds specifically to an epitope on an analyte. Similarly, a first member and second member of a binding pair specifically bind to each other, e.g., biotin and avidin and derivatives of biotin and avidin specifically bind to each other.

Detecting the presence of a target analyte in a sample may include providing a concentration of the target analyte. In certain embodiments, the detecting methods as disclosed herein may simply provide a “yes” or “no” answer. In other embodiments, the detecting methods may further provide an indication of the concentration of the target analyte.

Magnetic beads/particles used in the methods provided herein may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, NiO/Fe. Examples of ferrimagnetic materials include NiFe₂O₄, CoFe₂O₄, Fe₃O₄ (or FeO⁻Fe₂O₃). The magnetic beads may be substantially spherical and the size of the magnetic beads may selected based upon the amount of first binding element to be immobilized on the magnetic beads. The magnetic beads may be substantially solid with minimal or no pores traversing through the beads. In certain embodiments, the magnetic beads may have a diameter of 1 μm or more, such as 1-10 μm, e.g., 1-5 μm, 2-5 μm, for example, 2 μm, 3 μm, or 5 μm. In certain embodiments, the magnetic beads and dielectric beads used in the disclosed methods are provided in an amount sufficient to bind substantially all of the target analyte present in the sample. Since the magnetic and dielectric beads can be added in excess to the target analyte, the concentration of the target analyte can be determined from a concentration curve generated using the same beads and the same target analyte. Thus, in certain embodiments, in addition to detecting the presence or absence of a target analyte in a sample, the concentration of a detected analyte may also be determined.

Dielectric beads used in the methods provided herein may be substantially non-magnetic and may be substantially unaffected by magnetic field. In certain cases, the dielectric beads may be substantially non-conductive and may be unaffected by electric field. The dielectric beads may be substantially spherical and non-porous. The dielectric beads may be substantially opaque or substantially transparent. In certain embodiments, the dielectric beads may be colored, e.g., red, blue, green, yellow, neon, and the like so that they are easily visible to human eye. The different colored dielectric beads may be used for simultaneous detection of a plurality of analytes in a sample. For example, presence of a dielectric bead of a first visible color or a first optically detectable signature may be indicative of presence of a first analyte in a sample and presence of a dielectric bead of a second visible color or a second optically detectable signature may be indicative of presence of a second analyte in a sample. The size of the dielectric bead may be selected based upon the size of the nanoapertures in the array used to detect the presence of the dielectric beads. The dielectric beads are sized to be smaller than the nanoapertures (also called nanoholes). In some cases, the dielectric beads may have a diameter of at least 100 nm, e.g., 100 nm-1 μm, 200 nm-900 nm, 200 nm-700 nm, e.g., 200 nm, 300 nm, 400 nm, 500 nm, or 600 nm. Dielectric beads are made from dielectric materials such as, silica, polystyrene, glass, polypropylene, PTFE (Teflon), and polyethylene.

In various alternative embodiments, functionalization of the beads may be performed in any known standard manner. Modified bead surfaces may include carboxyl, amino, hydroxyl, and sulfates, pre-activated surfaces may include tosyl, epoxy, and chloromethyl groups, and bio-activated surfaces may include protein A, protein G, streptavidin-biotin. Modified bead surfaces provide a way to covalently attach a molecule such as antibodies. Functionalization of these surfaces could be done in a non-polar solvent. Pre-activated bead surfaces such as tosyl, epoxy, and chloromethyl groups add a level of control through manipulation of solution pH. Tosyl actively binds to sulfhydryl groups at a neutral pH, but switches to amino groups as the solution becomes more basic. An option is to use bio-activated surfaces of protein A/G and streptavidin-biotin affinity ligands. Streptavidin has an incredible specificity for biotin, and the affinity between the two is unaffected by changes in pH, salt concentration, or the presence of detergents. This specificity can be very useful. Another layer of control emerges in the fact that the link is reversible if the streptavidin-biotin conjugate undergoes a short 70° C. incubation. This feature introduces an easy method for the separation and removal of dielectric beads following target analyte isolation. The functionalization methods described above are performed by incubating the binding elements in molar excess with the magnetic or dielectric beads. In a particular example, magnetic beads may be functionalized with carboxylic acid which then allows conjugation of IgM or IgG antibodies in the presence of N-hydroxysuccinimide (NHS) and 1-Ethyl-3-(3-dimethylaminopropyl)-carbodiimide (EDC).

In certain embodiments, a magnetic bead may be functionalized for conjugating a first binding element to the magnetic beads via a covalent bond. In certain embodiments, a dielectric bead may be functionalized for conjugating a second binding element to the dielectric beads via a covalent bond.

In certain embodiments, a dielectric bead may be functionalized for conjugating a second member of a binding pair to the dielectric beads via a covalent bond. In certain embodiments, a second binding element may be conjugated to a second member of a binding pair via a covalent bond.

The array of nanoholes or nanoapertures may include a substantially planar substrate in which a plurality of through openings is present. An array may include at least 2 nanoholes. For example, an array may include 100-100,000 nanoholes, e.g., 1000-50,000, 10,000-50,000, 1000-50,000, 1000-5000, 1000-2000, or 500-1000 nanoholes. In some cases, the nanoholes may be arranged in a uniform manner, for example, a periodic arrangement of nanoholes disposed in a straight line, a circle, a square, or a rectangle and separated by a set distance that is constant. The diameter of the nanoapertures may be selected based the size of the dielectric beads. In certain embodiments, the diameter of the nanoholes may be up to 900 nm, e.g., 1 nm-900 nm, 10 nm-700 nm, 20 nm-500 nm, 50 nm-500 nm, 100 nm-500 nm, or 100 nm-300 nm. In certain embodiments, a planar substrate may include periodic array of suspended sub-wavelength nano-apertures (holes, with diameters between about 150-250 nm, or e.g. 10-250 nm, 50-250 nm, 100-250 nm, 100-125 nm, 100-300 nm, 150-300 nm, 150-200 nm or 175-225 nm or 200-300 nm or up to about to about 500 nm defined in a metal film with or without a supporting silica membrane (about 120 nm Au or e.g. about 80, 100, 125, 150, 200 nm) with a pitch length (e.g., center-center) about 500-700 nm, e.g. 100-1000, 250-1000, 250-900, 300-900, 300-800, 500-800, 500-700, 600-700 nm. In certain embodiments, a 50 μM×50 μM array may have 50,000-10,000 nanoholes.

Any suitable substrate may be used for making the array of nanoholes. In some examples, a dielectric substrate may be used. In some example, the substrate may be conductive or may be a dielectric substrate coated with a conductive material. The array of nanoholes may be of any suitable or convenient shape and size. In some embodiments, the array may be rectangular and may be 10 μm×10 μm, 10 μm×20 μm, 10 μm×30 μm, 10 μm×50 μm, 30 μm×30 μm, or 50 μm×50 μm, or no greater than 2500 μm², no greater than 1000 μm², no greater than 5000 μm², no greater than 7500 μm², no greater than 50000 μm², or no greater than 1 cm×1 cm.

The present methods utilize short incubation times. In certain embodiments, the time from introducing the sample and detecting the dielectric beads on the array of nanoapertures may be less than 1 min, less than 5 min, less than 10 min, less than 20 min, less than 30 min, or less than 60 min or less than 90 min, or less than 120 min.

Direct visual detection using the invention may include the use of a non-powered magnifying device such as a lens, magnifying glass or loupe, but in some embodiments it may explicitly exclude and does not require the use of any powered magnification device or method or image amplification or any additional external detector, e.g. an electronic detector, magnifier or image intensifier. In other embodiments, the signal is detected using powered detection, such as by using a photodetector, CMOS, camera, fluorescence detector, or colorimetric detector. Other embodiments may use electronic detection based on resistivity, impedance and current change.

### Devices

The methods disclosed herein may be performed using a number of devices, including single component devices or multicomponent devices configured for performing one or more of B2B conversion, dielectric bead enrichment, and detection of dielectric beads. The devices may be reusable or may include one or more components that are reusable.

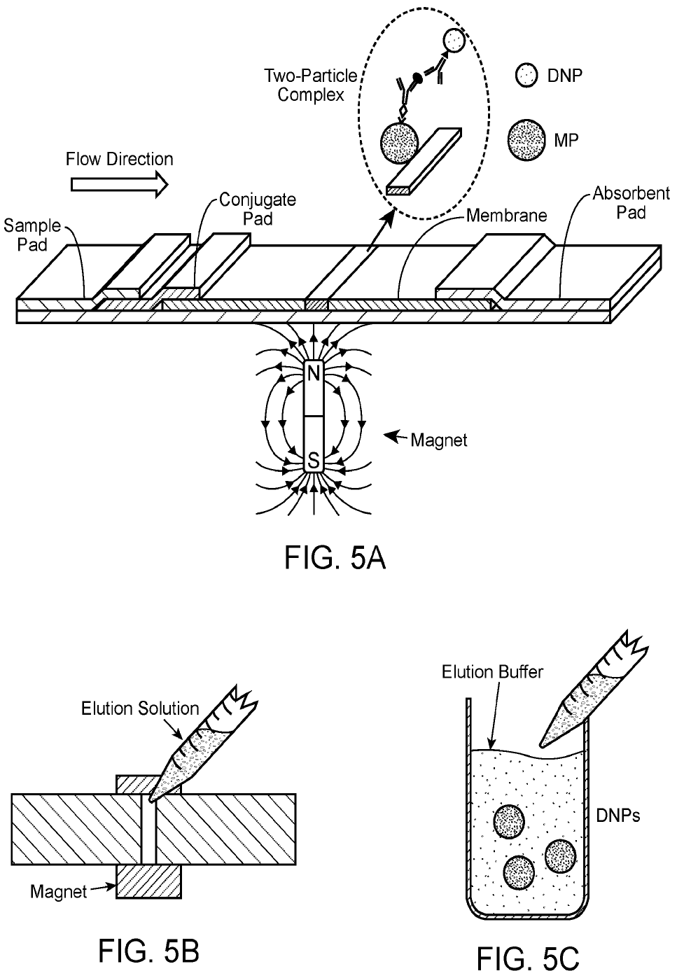

In certain embodiments, a device for carrying out the detection of presence of an analyte in a sample may include a lateral flow strip, such as, the device illustrated in FIG. 5A. In certain embodiments, the device may include a substrate comprising a first end comprising a sample loading pad disposed at the first end. The device may also include a reagent pad disposed downstream to the sample loading pad such that the sample flows from the sample loading pad to the reagent pad. The reagent pad may be loaded with magnetic beads that are functionalized to bind to the target analyte present in the sample as well as dielectric beads functionalized to bind to the target analyte. A magnet may be used to immobilize the magnetic beads such that any molecules/dielectric beads not bound to the magnetic beads flow downstream towards the second end of the device where an absorption pad is disposed. An elution buffer (e.g., containing reagents that dissociate the dielectric beads from the magnetic beads) may then be applied to the location at which the magnetic beads are immobilized at the magnet. See FIG. 5B. The elution buffer containing any dielectric beads may then be collected (e.g., by pipetting) and transferred for observation.

FIGS. 5A-5C illustrate a blueprint of a potential lateral flow assay approach for B2B conversion. FIG. 5A. Instead of functionalizing antibodies in a region of the substrate, a magnet would be used to capture two-bead complexes. FIG. 5B. An elution solution can be added and collected back using a pipette. FIG. 5C. The collected solution can be sent to a Naked Eye Detection chip (e.g., a nanohole array) for power-free diagnostics.

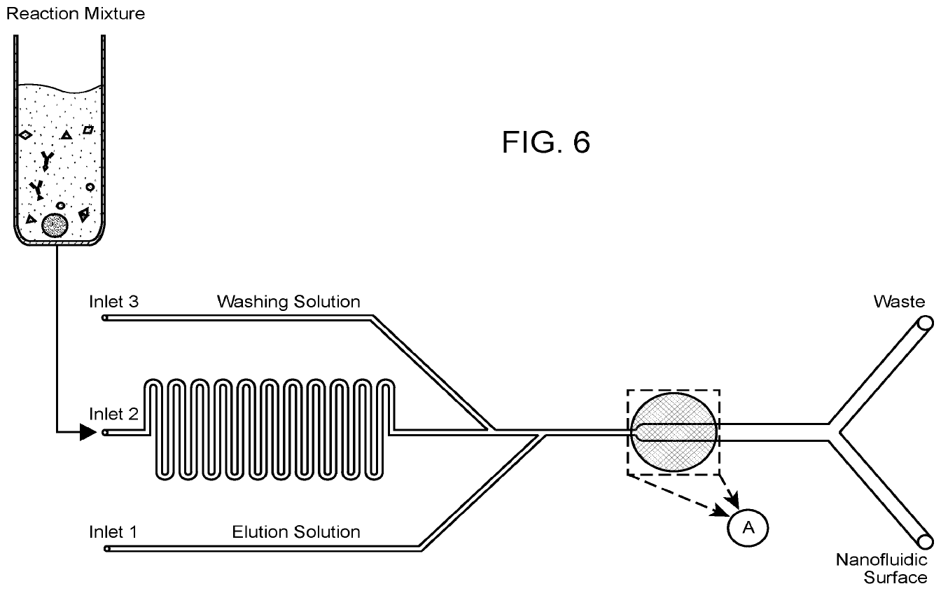

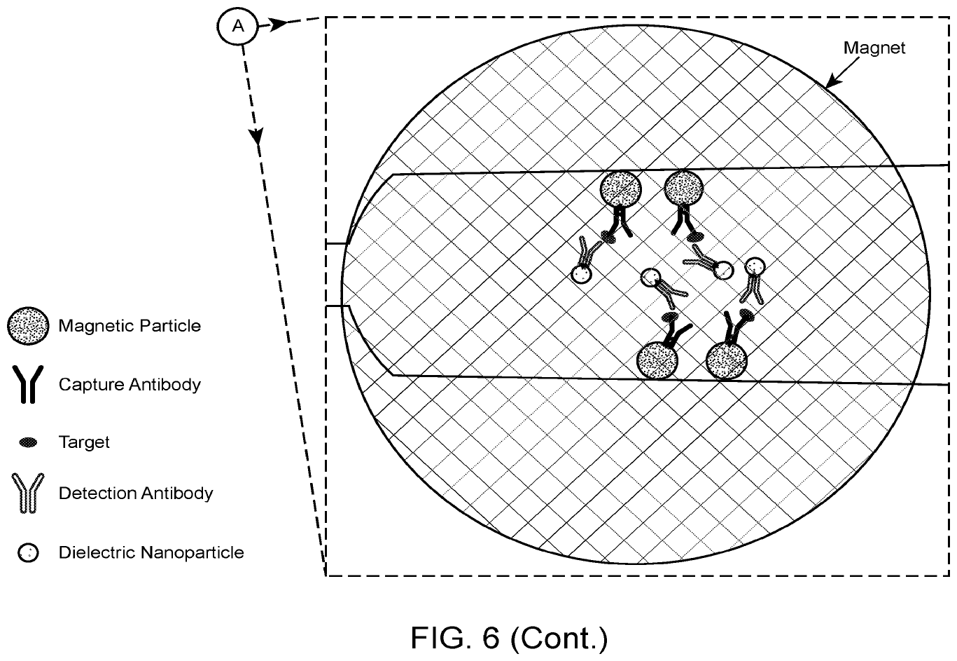

In certain embodiments, the presently disclosed methods may be partially or completely performed in a microfluidic device, such as, a device disclosed in FIG. 6. In certain embodiments, the device may include an elongated inlet where the functionalized magnetic beads, dielectric beads, sample, and first, second, third binding elements (if not previously immobilized on the beads) are mixed and allowed to interact to form two-bead complexes (magnetic beads bound to dielectric beads when the target analyte is present in the sample). The elongated channel may be shaped as a serpentine channel and may be connected to a channel at which magnetic field is applied to capture the magnetic beads and any two-bead complexes if present. This channel is also connected to first inlet for introducing a wash solution into the channel and optionally a second inlet for introducing the elution buffer into the channel. In some cases, the same inlet may be used for introducing the wash solution and the elution buffer. The channel may connected to one or more outlets for removing wash solution including any molecules not bound to the magnetic beads and thus not captured by the magnetic field. An outlet, e.g., an outlet for the elution solution may be connected to a collection device for collecting the eluted dielectric beads or to a device comprising an array of nanoapertures for detecting any dielectric beads present in the elution solution.

FIG. 6 The layout of a possible chip integrated version of B2B scheme is shown. It is envisioned that the sample solution could be mixed with nanoparticles, magnetic beads and antibodies in a vial before running the mixture through the “B2B Conversion Chip”. The diagram shows a reaction mixture that includes a target analyte and assay reagents, such as, magnetic beads; capture antibodies, where the magnetic beads and capture antibodies are functionalized to promote attachment of the capture antibodies to the magnetic beads; detection antibodies and dielectric beads, where the dielectric beads and detection antibodies are functionalized to promote attachment of the detection antibodies to the dielectric beads, flowing into the microfluidic system from Inlet 2 and washing towards the waste output. As noted herein, in some assay formats, the detection antibody may be attached to the dielectric beads using a two-part high affinity binding couple. While the mixture is flowing along the serpentine shaped channel, two-particle complexes would be created (if the target molecules are present). The two-particle complexes and the freely floating magnetic beads could then be collected at the channel walls in the magnetic field region indicated by a disk shaped magnet. Non-magnetic nanoparticles and biomolecules would be washed away to the waste. To remove any non-specifically captured non-magnetic molecules from the B2B Conversion Chip, a washing solution (PBS) would be introduced from the Inlet 3 and would flow towards the waste. Finally, an elution solution would be introduced from Inlet 1 as the waste outlet is closed and the outlet connecting to the Nanofluidic Chip containing a nanohole array is opened. The elution solution will dissociate the two-particle complex and nanoparticles would be released into the elution solution. Finally, the nanoparticles would be collected at the detection surface using flow through approach. B2B Conversion Chips and Nanofluidic chips can be fabricated by irreversibly bonding molded polydimethylsiloxane (PDMS) channels to a 3×1 inch glass slide. Valves may be included at appropriate regions to control fluid flow.

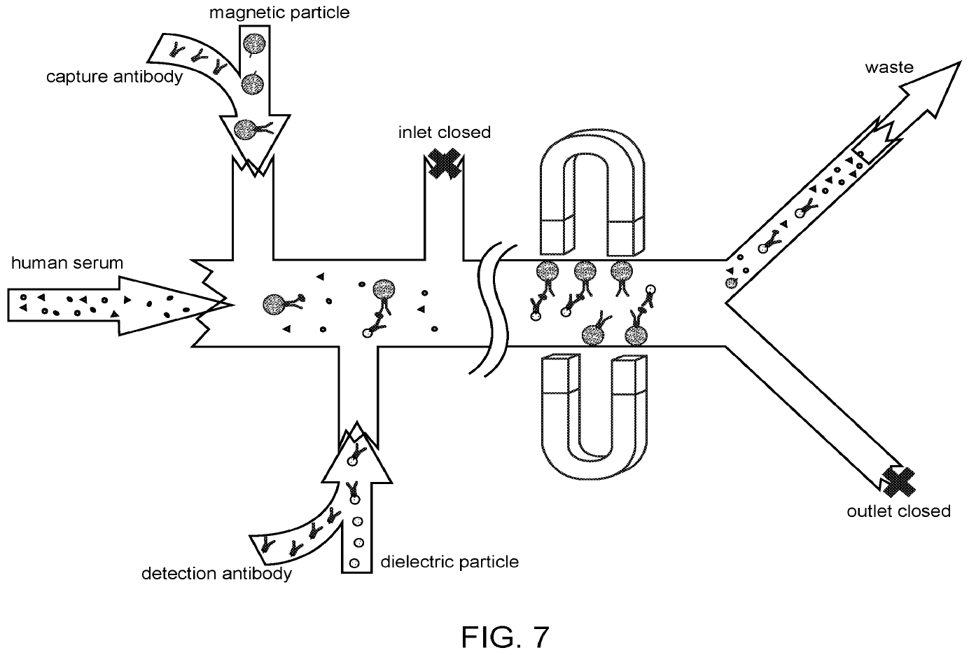

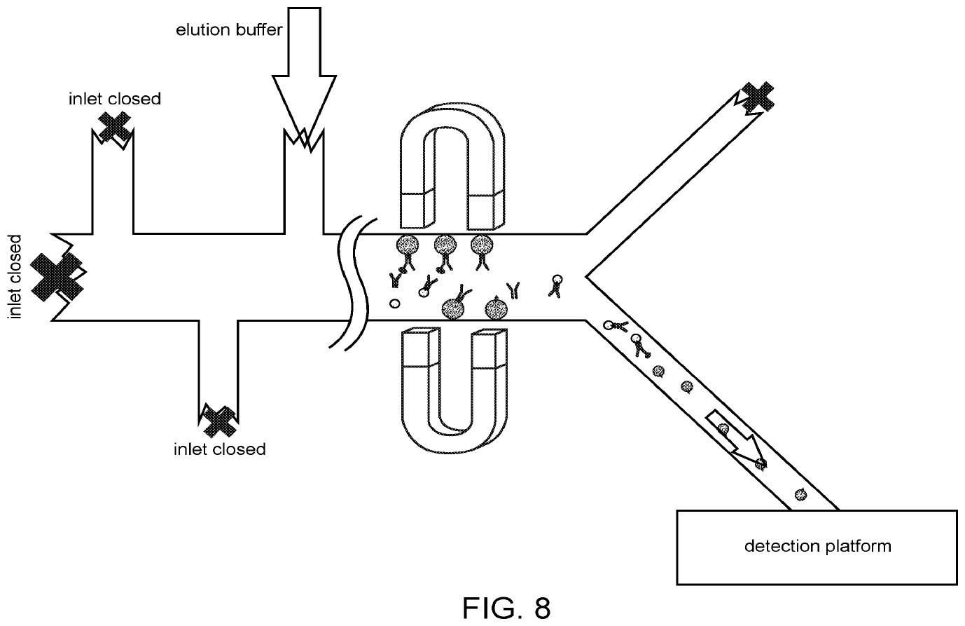

In an alternate embodiment, a device for B2B conversion may include three separate inlets as shown in FIGS. 7 and 8 for simultaneously introducing a sample (e.g., serum or blood), magnetic beads functionalized with a first antibody (e.g., a capture antibody) that binds to the target analyte present or suspected of being present in the sample, and dielectric beads functionalized with an antibody (e.g., a detection antibody) that also binds to the target analyte. The detection antibody may be attached to the dielectric beads directly or via a two-part high-affinity binding couple where the detection antibody binds to a first member of the two-part high-affinity binding couple and the dielectric bead is coated with a second member of the two-part high-affinity binding couple. The incubation may occur in a microfluidic channel that may initially be closed such that fluid is not flowing out of the channel. Following sufficient incubation time to allow formation of a two-bead complex, a magnetic field is applied to the channel to capture the magnetic beads and the channel opened to allow fluid flow out of the channel and to a waste reservoir (FIG. 7). After any optional washing of the captured magnetic beads, a separate inlet may be used to introduce the elution buffer and elute dielectric particles moved to a nanohole array for enrichment and detection (FIG. 8).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Comparison of Nanofluidic Enrichment and Conventional Microfluidics

An elution solution comprising 200 nm dielectric beads at a concentration of 10⁸ beads/ml was run through the nanohole array at a rate of 10 μl/min (FIG. 3B). Optimal accumulation of the dielectric beads was observed using a vertical flow scheme involving nanohole openings projecting through suspended membranes. When the same solution was allowed to run through the channel using a conventional flow scheme (flow over)—where the elution solution follows parallel to the surface—less accumulation of the nanoparticles was observed. These results indicate that it is easier to capture the beads at high efficiencies on channel walls using vertical flow.

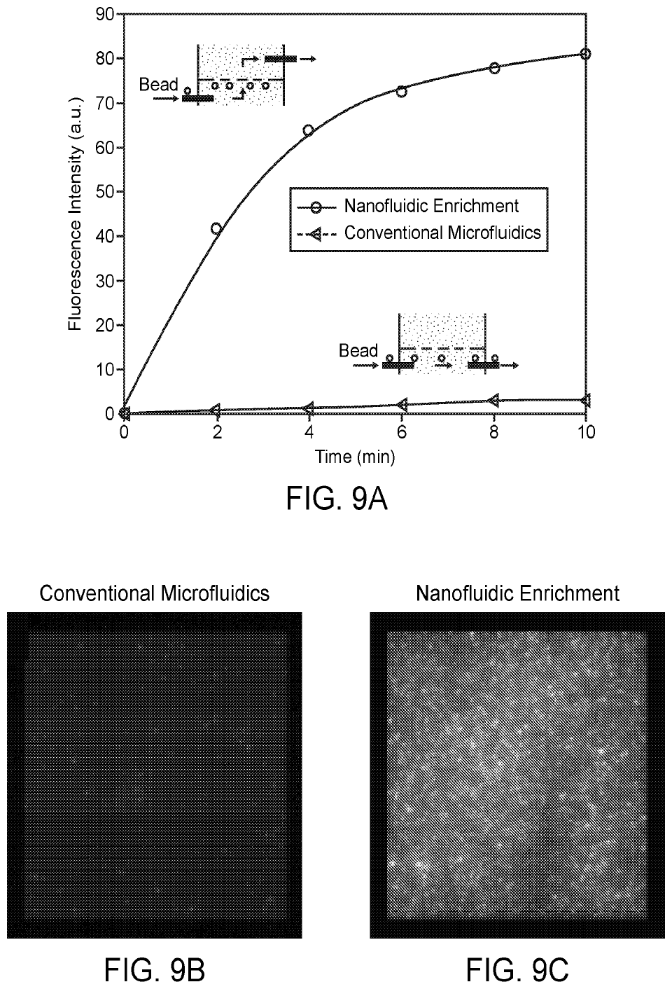

In another experiment, the biomarker (target analyte) detected was prostate specific antigen (PSA).” In addition, “biomarker-to-bead conversion” is interchangeable with “biomarker-to-bead transformation” or “B2B conversion” or “B2B transformation.”) FIGS. 9A-9C illustrates the fundamental mass transport limitation is overcome by transporting the eluted dielectric beads towards the nanohole surface. A 100 aM concentration of PSA spiked in 50% pooled human serum was B2B converted into 500 nm dielectric beads. As the PSA concentration in the experimental sample was extremely low and the eluted dielectric particle concentration is approximately on the same order of magnitude as the target PSA concentration, randomized diffusive transport of the dielectric beads to a detection sensor would require unrealistically long incubation times typically extending from hours to days. By flowing the eluted dielectric beads through a suspended nanohole array in a gold coated silicon nitride membrane, the convective streamline of fluid carrying the eluted dielectric beads can be directly diverted towards the nanohole array surface. The cross flow efficiently brings the dielectric beads towards a nanohole array surface with an area of less than 1 mm². The diameter of the nanoholes is 200 nm—smaller than the 500 nm diameter of the dielectric beads. As a result, the dielectric beads accumulate on the nanohole array surface as shown in the SEM image. Effectively zero dielectric beads accumulate on the nanohole array surface under conditions allowing random diffusive transport as depicted in FIG. 9B.

The number of accumulated dielectric beads can be quantified using dielectric beads labeled with green fluorescent protein (GFP) or a similar fluorescent protein or other fluorescent label by measuring the fluorescence intensity within the membrane window as the dielectric beads accumulate. Real-time physical accumulation of dielectric beads can be measured by an increase in fluorescent intensity. As shown in FIG. 9A, the fluorescent intensity is continuously increasing for a time span of about 10 min at a flow rate of 20 μl/min. It was observed that the fluidic flow was not affected by the clogging of the nanoholes using the cross flow method. Additionally, negligible fluorescent intensity change was observed using a convective diffusive flow method (FIG. 9B) compared to significant fluorescent intensity alteration in cross flow regime (FIG. 9C) after 10 minutes of flow.

FIG. 3A is an SEM image of a suspended nanohole (nanopore) array. Nanoholes (also called nanopores in this disclosure) serve both as optical nano-sensors and nano-fluidic channels.

FIG. 3B. Enhanced mass transport is shown using a nanofluidic enrichment approach (“Flow through”) as compared to a convective microfluidic approach (“Flow over”). The resonance shift is due to the protein accumulation on the sensing surface.

FIG. 9A. The mass transport limitation was overcome using cross flow-enrichment of dielectric beads on the sensor surface was enhanced compared to the convective flow approach. The curves for nanofluidic enrichment and conventional microfluidics represent the cross flow and convective flow schemes, respectively. FIG. 9B. Negligible fluorescent intensity was observed under convective flow “Flow Over”. FIG. 9C. Dielectric beads were efficiently enriched on the sensor surface by nanofluidic enrichment (“Flow through”) as indicated by the considerable fluorescent intensity within the membrane window.

### Example 2: Visual Detection of Biomarkers Due to Resonance Shift

A metal (Au) film of about 120 nm in thickness with a pitch length of about 500-700 nm was used. The film also included nanohole array (NHA) sensors—a periodic array of suspended sub-wavelength nanoapertures (holes with diameters of about 150-250 nm). Spectral responses upon accumulation of dielectric beads could be observed using three-dimensional (3-D) Finite Difference Time Domain (FDTD) electromagnetic simulations. The film was designed to be optically thick and the nanoapertures of such a diameter that they were too small to transmit light. Thus, incident light could then only be transmitted at specific resonant wavelengths via an optical process incorporating surface plasmon polaritons (SPPs). Biomolecules/pathogens binding to the metallic nanohole surfaces increased the effective refractive index of the medium around the nanoholes, which led to red shifting of the plasmonic resonances. Furthermore, measurement of the Fano resonance profile resulting from the readings of the NHA sensors, resulted in visual confirmation of the capture of target biomarker proteins on the NHA sensor surfaces with the naked eye using a nanohole array with an area of 90 micrometers by 90 micrometers. A band-pass filter (FWHM=10 nm at λ=670 nm) spectrally tuned to the plasmonic resonances peak filters the incoherent broadband light outside of the resonant transmission peak of the Fano resonance. When the IgG antibodies bound to the protein A/G that are immobilized on the surface, the accumulation of said antibodies resulted in an increase in the effective refractive index of the nanoplasmonic surface, which in turn resulted in a red shift, or a resonance shift (This was also measured by 3-D FDTD simulations.) As more proteins accumulated on the sensing surface, the resonance shift increased and became more pronounced. This experiment was done in ordinary laboratory settings without light isolation, and in these settings, the intensity change was sufficient to discern with the naked eye. Less than 30 seconds were required for the operator to tell if the target biomarkers were present in the sample solution using visual inspection. See Yanik, PNAS, Jul. 19, 2011, vol. 108 no. 29, 11784-11789; incorporated by reference herein.

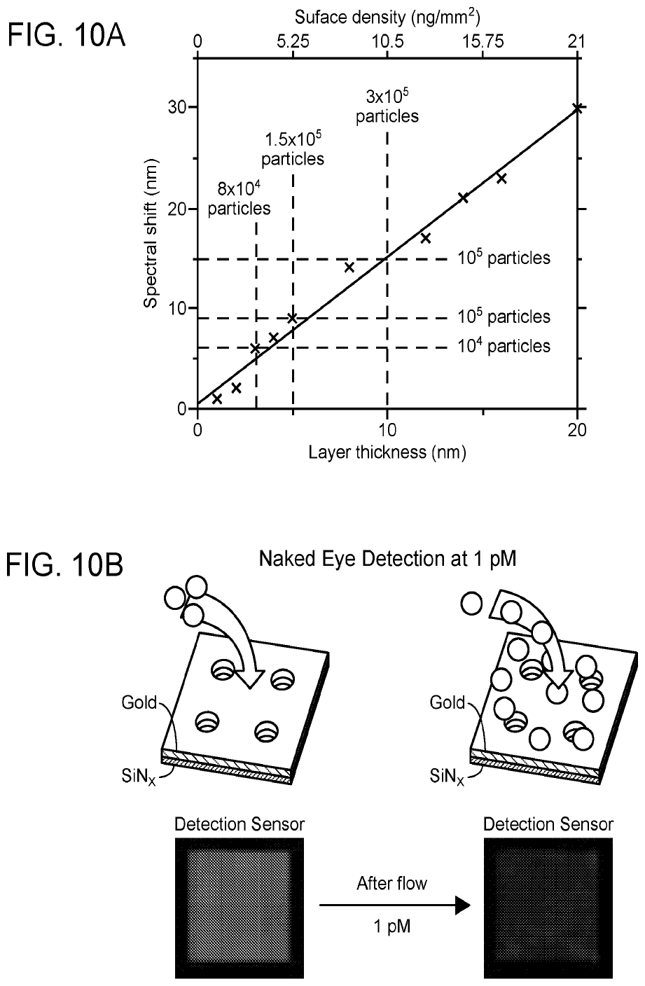

In calibration experiments, known concentrations of dielectric beads were spiked into pure solutions and the resonance shifts compared after flow through the nanohole surfaces. The experimental measurements and FDTD numerical models showed strong agreement. Using this calibration data, it was estimated that as few as 100 dielectric beads could be detected in plasmonic nanohole sensors using a handheld spectrometer. See FIG. 10A.

An experimental demonstration of this naked-eye detection technique with end-point measurements is shown in FIG. 10B. Initially, dielectric beads were isolated from a physiological solution (human serum) spiked with 1 pM target biomarker (PSA). After running the dielectric beads in elution solution through the NHA sensors, accumulation of the dielectric beads elicited a large enough red shifting of the plasmonic resonance to result in spectral overlapping of the transmission minima (Wood's anomaly) of the nanohole array with the transmission window of the band-pass filter. Accordingly, a dramatic reduction in the transmitted light intensity (defined by the band-pass filter at λ=680 nm) was observed upon capturing of the dielectric beads as shown in FIG. 10A. As illustrated herein, this nano-biophotonic approach does not require fluorescence agents, enzymatic reactions, chemical amplification processes, optical apparatus (lenses, objectives, etc.) and power-operated electronic instrumentation (light sources, photodetectors, cameras, etc.) to operate. It further offers a limit of detection that is better than the most sensitive enzyme-linked immunosorbent assays (ELISAs) but can be performed in significantly less time.

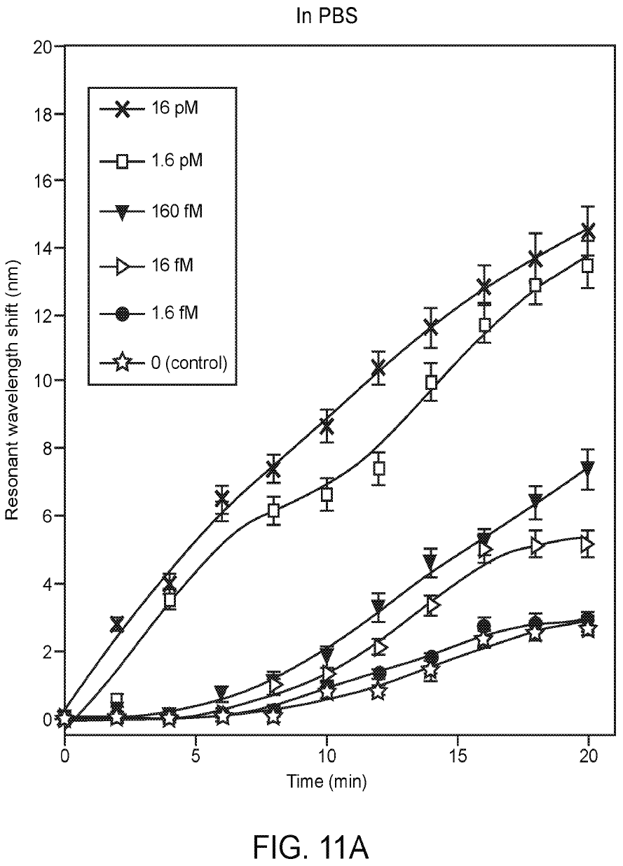

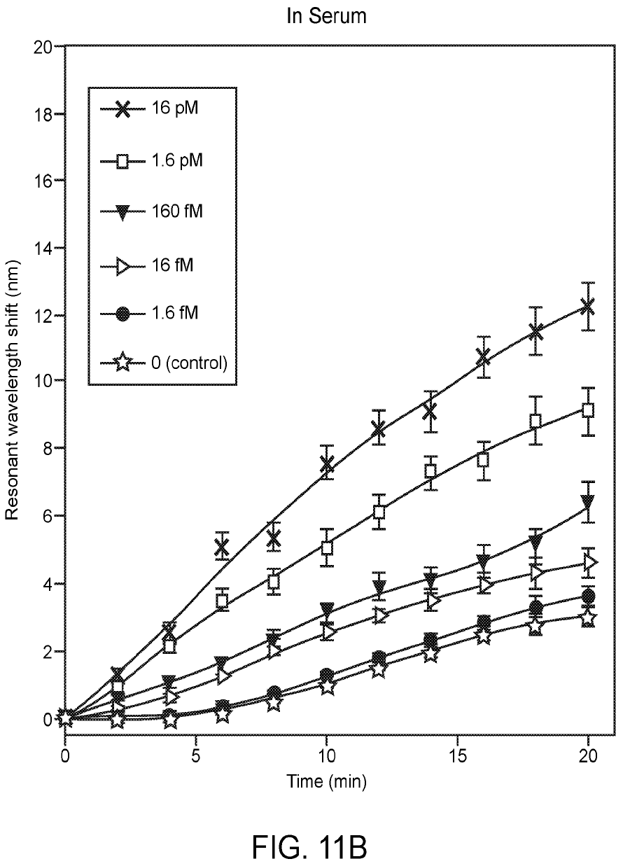

In another example, varying concentrations of a target antigen (PSA) are analyzed using the disclosed methods and devices. After the B2B conversion scheme and flowing the elution solution including the dielectric beads through the nanohole array, (at a 5 μl/min flow rate) the resonance wavelength shifts are measured in real time (where the x axis indicates the time of measurement). Experiments are repeated for controlled concentrations of targets in PBS (FIG. 11A) and Serum (FIG. 11B). The resonance shift in the control sample (without PSA) is indicated as the control sample. It was observed that 1.6 fM concentrations of PSA were still detectable because the resonance shift of the experimental sample was larger than that of the control sample.

FIG. 10(a) Theoretical, 3-D FDTD and experimental analysis was performed to understand EOT resonance behavior upon accumulation of DNPs. As the number of accumulated particles increased, the layer thickness increased, and as observed, the spectral shift increased as well. (b) For biomarker concentrations of 1 pM, the B2B conversion scheme yielded enough accumulated dielectric beads such that the result was discernible to an unaided human eye.

FIG. 11 shows an embodiment of the detection scheme for varying concentrations of a target antigen (PSA). After performing B2B conversion and running the elution solution (at 5 μl/min flow rate) through the nanohole array, the resonance wavelength shifts were measured in real time (x axis indicated time of measurement). The experiments were repeated for known concentrations of PSA in PBS (FIG. 11A) and Serum (FIG. 11B). The resonance shift in the negative control sample (no added PSA) is indicated as the control sample. It was observed that the resonance shift was discernable over the negative control in a sample with a 1.6 fM concentration of PSA.

### Example 3: Biomarker-to-Bead (B2B) Conversion

B2B conversions were performed as outlined in FIGS. 1 and 2.

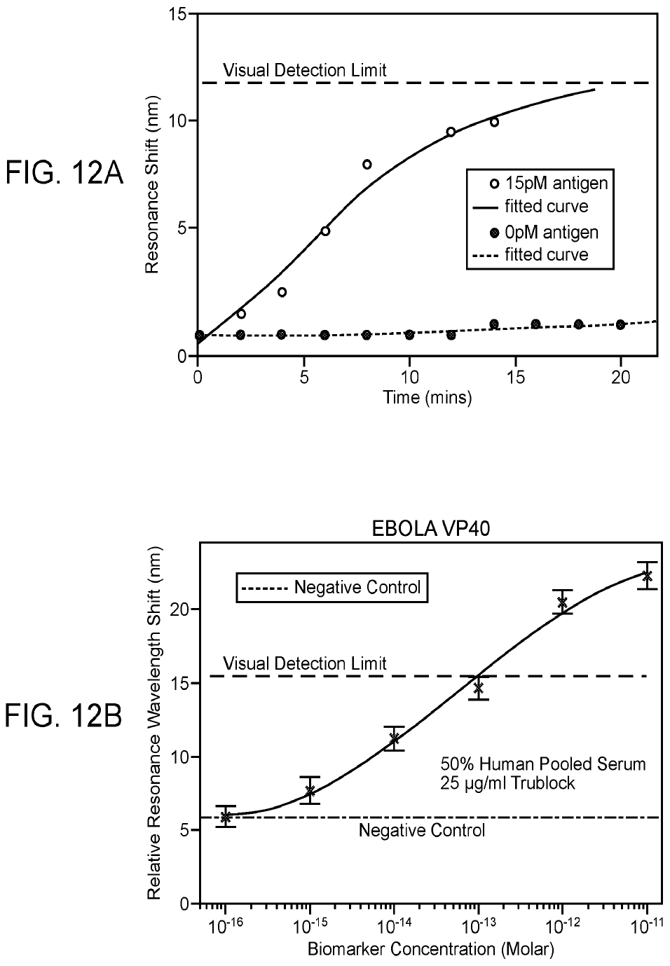

Visual detection was demonstrated for target analyte concentration of 15 pM. A commercial ELISA kit (CMC4033, Thermo Fisher Scientific) was used for comparison. Initially, a mouse anti-IFN-γ antibody (6 μg/ml) was incubated with Superparamagnetic Dynabeads (2.8 μm diameter, 0.75 mg/ml concentration—Thermo Fisher Scientific) for surface conjugation. After a magnetic wash and resuspension, the antibody-conjugated magnetic bead solution was mixed with 10 μL stock solution containing mouse IFN-γ at 250 pg/ml. After 20 mins, a biotinylated anti-mouse IFN-γ secondary-antibody was added. Subsequently, NeutrAvidin coated silica beads (200 nm diameter) were added and incubated for an hour at room temperature using a Hulamixer (following the ELISA kit protocol optimized for titter plates). A magnetic separator was used to pellet the magnetic beads, the supernatant was removed, an elution buffer added and then incubated for 2 mins to release the captured silica beads from the immobilized magnetic beads. The elution buffer containing free silica-beads was directed onto NHAs using the disclosed cross flow method at a rate of 5 μl/min. Quantitative measurements of silica bead accumulation on the biosensor surfaces was performed using the spectral analysis of the transmission (EOT) signal (FIG. 12A). A spectral shift of ˜12 nm, which is sufficient for visual detection (Yanik, PNAS, Vol. 108 (29): 11784-11789 (2011)), was observed within 20 mins. In negative control experiments, the procedure was repeated without mouse IFN-γ antigen; a resonance shift<1 nm was observed.

In another example, the target protein (term interchangeable with “biomarker” or “target analyte”) was an Ebola VP40 glycoprotein, and the sample solution was 10-20 microliters of human blood. A trial was run in which Ebola VP40 glycoproteins were added into human blood at a concentration of 100 aM. A negative control was run in tandem. Magnetic beads with diameters of 2.8 micrometers were functionalized with IgG antibodies that bind to Ebola VP40 glycoproteins, mixed with the human blood sample solution and incubated. A magnetic field was then applied (using a magnet), pulling the beads away from the sample solution. The non-specific molecules in the sample solution were then washed away using a washing solution. Subsequently, the magnetic beads were re-suspended, mixed with biotinylated IgM antibodies that bind to Ebola VP40 glycoproteins and incubated. The magnetic bead complex was pelleted using a magnetic field, and the non-bound IgM antibodies were washed away. The magnetically captured beads were suspended and subsequently incubated with NeutrAvidin functionalized silica (dielectric) beads, which were 200 nm in diameter. The magnetic beads were pelleted again by the applied magnetic field, and the non-specific dielectric beads were washed away. While still immobilized by the magnetic field, an elution solution was run through the mass of bead complexes to disassociate the dielectric beads from the magnetic beads. The elution solution was then transferred to the detection device (in this case, a suspended nanohole array) for visual evaluation.

The trial in which the Ebola glycoproteins were present resulted in the accumulation of silica beads at the suspended nanohole array. The negative control resulted in significantly fewer beads accumulating, due to non-specific interactions. The entire process took less than 25 minutes.

The dilution curve for the above experiment resulted in a limit of detection (LOD) around 100 aM. Experiments were repeated three times for each concentration point in FIG. 12B.

FIG. 1 illustrates an embodiment of a biomarker to bead conversion method disclosed herein followed by enrichment for the dielectric beads that were bound to the target analyte bound to the magnetic beads. The dielectric beads attached to target analyte bound to the magnetic beads are dissociated and the solution containing the dissociated dielectric beads transferred through the nanoapertures of an array. The array may be housed in a device comprising a chamber divided by the array into a first chamber and a second chamber. The first chamber may be include an inlet for introducing the dissociated dielectric beads into the first chamber and the second chamber may include an outlet for removing the dissociation solution. The first chamber may include an open top to facilitate visualization of any dielectric beads captured on the surface of the array. Alternatively the top of the first chamber or at least one wall of the first chamber may be substantially transparent to facilitate visualization of the surface of the array. The surface of the array on which the dielectric beads are trapped may be referred to as the top surface. The approach outlined in FIG. 1 combines biomarker-to-bead conversion, surface enrichment and naked eye detection to achieve detection limits as low as 1 pM. The total assay time is estimated to be <30 mins.

FIG. 2. Biomarker-to-Bead (B2B) Conversion Scheme. The sample is incubated with functionalized magnetic and dielectric nanoparticle beads and secondary antibodies. A magnetic field is used to pellet the two-particle complex created through the sandwich assay. An elution solution neutralizes the proteins and antibodies. The final elution buffer contains the dielectric nanoparticle (DNP) beads in excess of a negative control if target biomarkers are available in sample solution. SEM images are presented for 1 fM biomarker concentrations and negative controls. An insignificant (near zero) number of DNP beads are carried to the final elution solution in negative controls. Experiments were performed using Ebola antigens spiked in buffer solution and two complementary antibodies designed for enzyme-linked immunosorbent assay tests.

FIG. 12A. In controlled experiments with mouse IFN-γ antigens, strong resonance shifts of ˜12 nm were observed (visually discernible through Fano Resonances) for 15 pM target concentrations. Minimal resonance shifts (<1 nm) were observed in negative control experiments. The visible intensity change was not due to the optical blocking of nanoholes by the beads. The accumulated effective mass on top of the nanohole array caused the resonance wavelength shift, hence the intensity change. The optical signal would not have been affected by where the beads are located on the surface even if they were inside the openings. The nanoparticles were transparent and would have been too small to create any strong scatterings or block the transmitted optical light.

FIG. 12B. Quantitative and repeated measurements of EBOV VP40 antigens in 50% human serum at ˜100 aM levels were demonstrated in this figure. The negative control and visual detection limit are marked.

### Example 4: Detection of Biomarker Using B2B Conversion and Electrochemical Cell

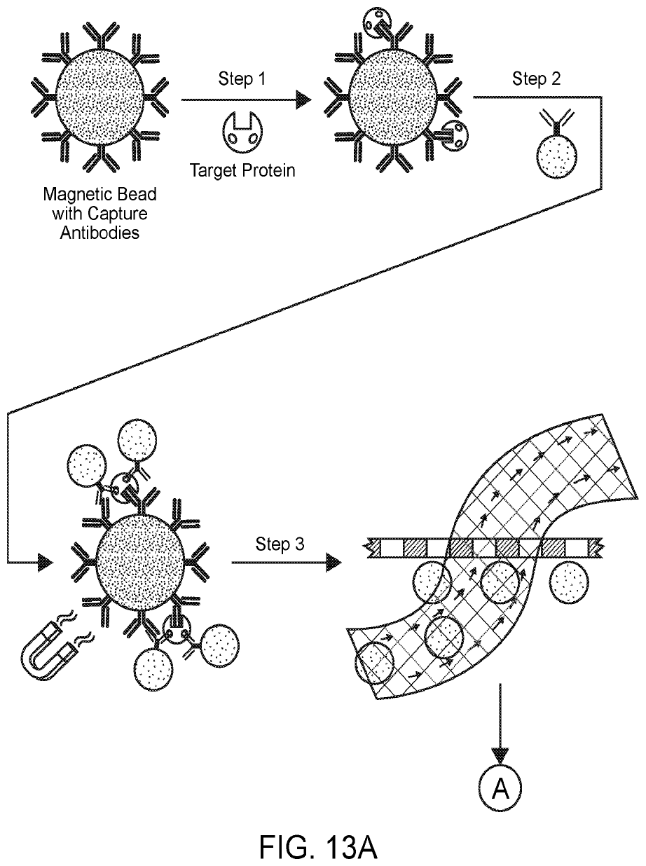

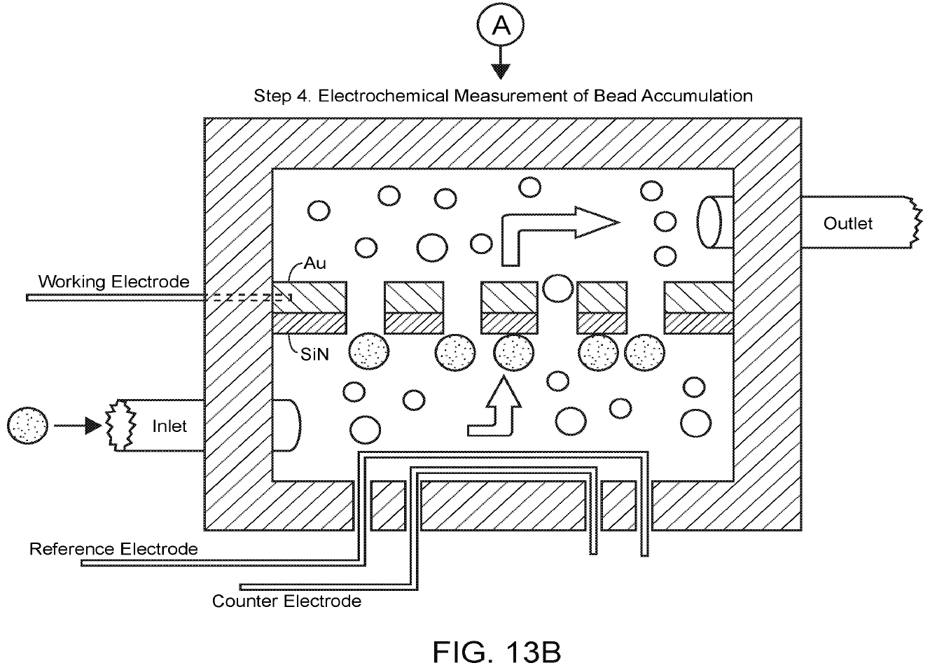

FIG. 13A illustrates protein detection and quantification schemes. Creation of a magnetic/dielectric two-bead complex enables specific biomolecular recognition using a pair of antibodies that bind to two different epitope sites of the target protein. The target proteins can be isolated from pooled human serum, captured in the two-bead complex, and the dielectric nanoparticle beads eluted. As a result, the concentration of the target protein directly correlates to the number of dielectric nanoparticle beads. In other words, the target protein is converted to a nanoparticle surrogate. The nanoparticles are enriched onto a suspended nanohole array based electrochemical sensor through microfluidic delivery. The nanohole array, constructed by depositing gold onto the suspended silicon nitride membrane with a prefabricated nanohole pattern, not only operates as a conduit for fluidic transportation, but, through its gold surface also operates as the detection electrode. The freestanding nanohole array is mounted in a multilayered microfluidic system, where the addition of a fluidic inlet and outlet control the fluidic streamline.

By incorporating reference (Ag/AgCl) and counter (platinum (Pt)) electrodes in the bottom fluidic compartment, a complete electrochemical cell is constructed (FIG. 13B). A current-voltage response of this electrochemical cell is obtained through a non-Faradaic process—the movement of electrolytic ions. The physical adsorption of dielectric beads limits the chemical ion transport and thus triggers an impedance rise in the electrochemical system, which leads to a drop in current signal at a fixed voltage. This magnitude of the drop in signal correlates with the concentration of nanoparticles, as well as the target protein concentration.

FIG. 13B provides a schematic of an electrochemical cell used for electrochemical detection of the target analyte by detecting impedance of ionic current caused by occlusion of the nanoapertures by the dielectric beads. The electrochemical cell included a first chamber connected to an inlet and a second chamber separated from the first chamber by an array of nanoapertures. A counter electrode and a reference electrode are disposed in the first chamber. The array of nanoapertures includes a coating of a conductive material facing the second chamber. The coating of conductive material serves as a working electrode in the second chamber. An outlet is also provided in the second chamber. As shown in FIG. 13B, elution solution (containing dielectric beads) introduced into the first chamber via the inlet flows through the nanoapertures into the second chamber and exits via the outlet. The dielectric beads occlude the nanoapertures leading to a drop in ionic current flowing across the nanoapertures and into the second chamber.

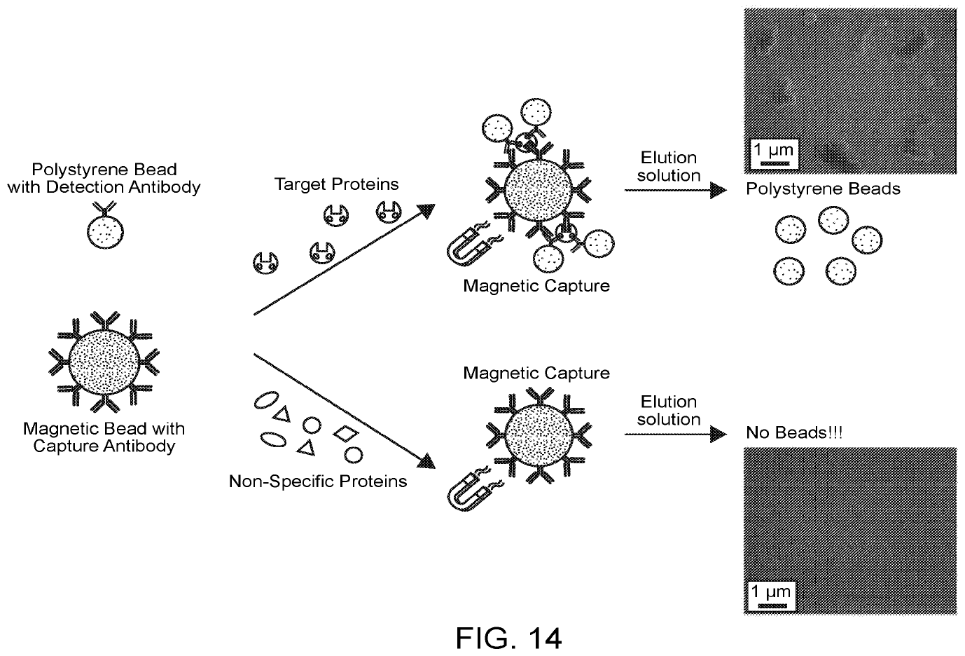

The magnetic bead, capture and detection antibodies (anti-prostate specific antigens), target antigens (prostate specific antigens (PSA)), and dielectric beads are acquired from commercially available vendors. To detect PSA, 12 μg of capture antibody 2H9 was covalently coupled to 1 mg of the magnetic bead (Dynabeads M-270 Epoxy, 2.8 μm) in a final volume of 1 ml in phosphate-buffered saline (PBS, purchased from FisherScientific) following the coupling protocol provided by ThermoFisher. Then the coupling solution was divided into ten equal parts with each part 100 μl in volume in a microcentrifuge tube. All the tubes were placed on the magnet for 2 minutes and the supernatant was pipetted off after the magnetic bead was translocated against the tube wall just before adding PSA samples. PSA were prepared by diluting the stock solution in 50% pooled human serum (Pooled Normal Human Serum, purchased from Innovative Research) mixed with 25 μg/ml active blocking agent (TRU Block from Meridian Life Science). A 500 μl volume of each sample with was incubated with anti-PSA antibody conjugated magnetic beads with end-over-end rotation at room temperature for 20 minutes, followed by incubation with a 500 μl volume of biotinylated anti-PSA detection antibody (5A6) at a concentration of 0.6 ug/mL with rotation at room temperature for 20 minutes. Magnetic beads complexed with detection antibody were magnetically separated from the free detection antibody and washed gently with 1 ml PBS buffer 3 times. Subsequently 500 nm sized avidin-labeled dielectric beads at a concentration of 10⁸/ml were added to the complex and incubated with rotation at room temperature for minutes. Complexes including the antibody conjugated magnetic beads, PSA, biotinylated detection antibody, and avidin-conjugated dielectric beads were purified magnetically from unbound dielectric beads by washing the assay with 1 ml PBS buffer 3 times and the supernatant was removed immediately after the washing was done. A 1 ml volume of elution buffer (50 mM Glycine pH 2.8), was added into each tube and incubated with the final coupling assay with rotation for 10 minutes to dissociate the dielectric beads, and then the supernatant containing dielectric beads was transferred into a new tube. A negative control experiment was also conducted following the exactly same procedure but using pure 50% pooled human serum mixed with 25 μg/ml active blocking agent without any PSA. Scanning electron microscope (SEM) images were taken of a 6 μl of final elution solution drop cast on a silicon substrate. Dielectric bead concentration and incubation time parameters were further optimized using SEM measurements. Dielectric beads with comparable surface densities were recovered when using the procedure with samples containing PSA. See FIG. 14.

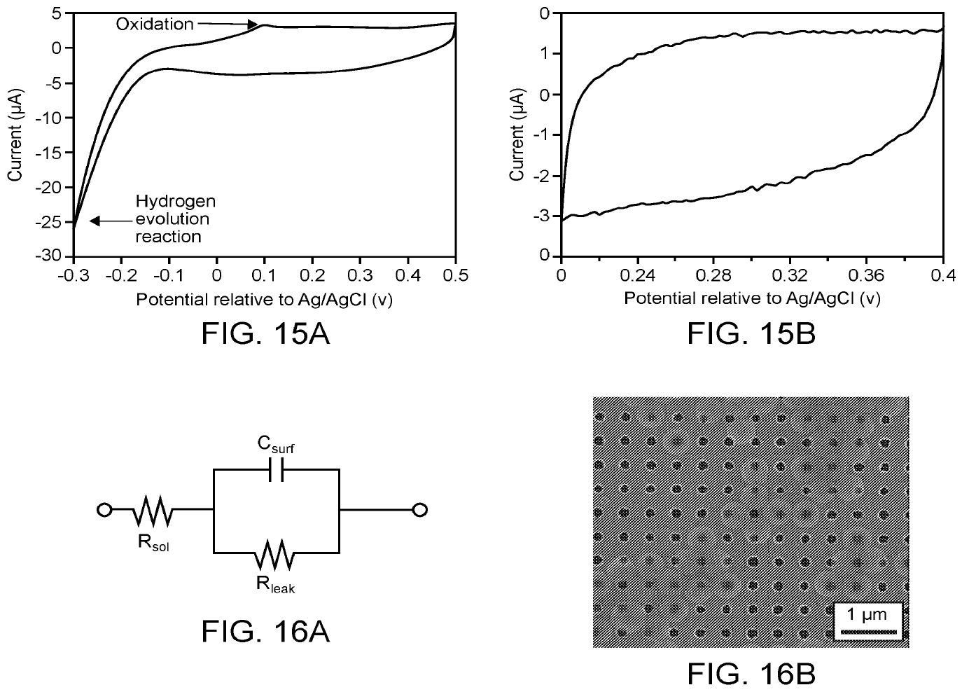

FIGS. 15A and 15B illustrate the cyclic voltammetry (CV) measurement to determine a proper scanning range for the applied potential for square wave voltammetry (SWV) measurement. CV reveals the presence of a redox reaction hence is useful for the determination of the potential window where no redox reactions can take place on the gold film. With this technique, the interference of Faradaic reactions can be greatly reduced and therefore excluded in the SWV measurement. Any current change in SWV is mainly due to the alterations in the non-Faradaic process—the movement of electrolytic ions across the nanofluidic channels. As shown in the FIG. 15A, hydrogen evolution was activated at −0.3V, as the potential rises, the gold film started to experience an oxidation peak at 0.1 V that then plateaued at potentials greater than 0.1 V. In order to prevent these undesirable reactions from occurring, a potential window within the 0.2 to 0.4 V was chosen. The approximately rectangular loop shown in FIG. 15B indicated the non-existence of Faradaic reactions on the gold film in the scanned potential window. The observed current is governed by electrokinetic transport of electrolytic ions through the nanoholes.

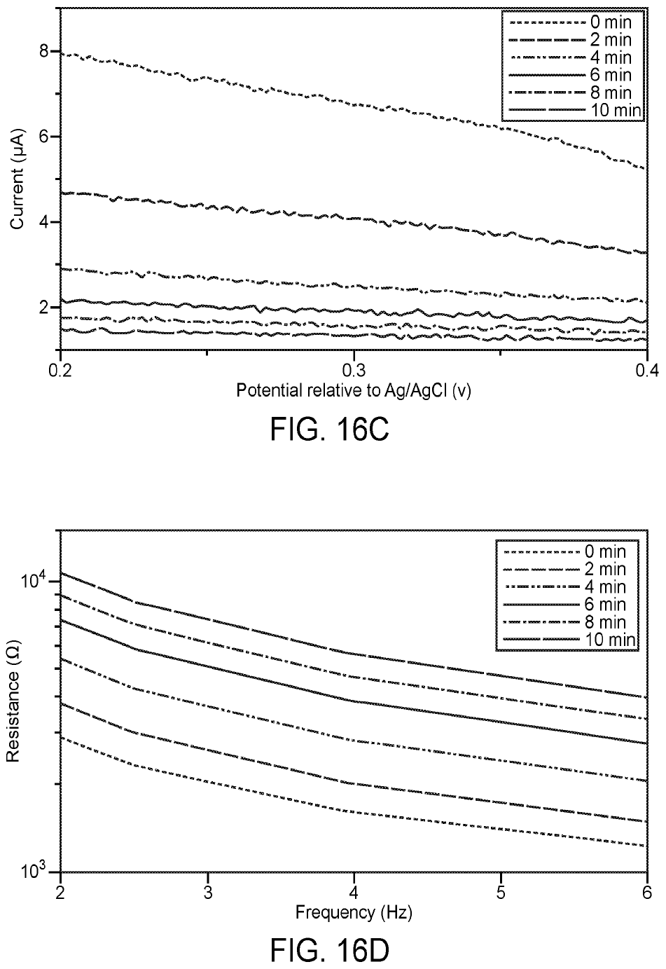

FIGS. 16A-16D illustrate the quantification of 100 aM of PSA by electrochemical methods including square wave voltammetry (SWV) and electrochemical impedance spectroscopy (EIS). After B2B converting the 100 aM of PSA into corresponding dielectric beads, the beads were accumulated onto the chip—SiNx surface by nanofluidic enrichment at a flow rate of 20 μl/min for 10 min. Within this time window, the SWV and EIS measurements were taken every 2 min while keeping the bead solution flowing through the nanoholes. As more nanoholes are plugged up (shown in FIG. 16B) by the larger sized dielectric beads, there is less fluidic passage for ions to move, as a consequence, less current is generated in the electrical loop, and correspondingly the impedance becomes larger. This nanohole array based electrochemical cell is electrically equivalent to a simple circuit model shown in FIG. 16A, where C_{surf} is the capacitance established at the gold-solution interface, Rₗₑₐₖ depicts the resistance of the nanoholes through which ionic movement from bulk solution to gold, and Rₛₒₗ is the resistance of the bulk solution in the electrochemical cell. Since the current and impedance alterations in the electrochemical cell are mainly caused by the suppression of ionic movement by occluding the nanoholes, Rₗₑₐₖ is the primary part that accounts for the signal change and thus can be used to quantify the concentration of the dielectric beads. In order to extract values of Rₗₑₐₖ from EIS measurement, applied ac potential is swept from 2 to 6 Hz to eliminate the capacitive component. Here the EIS is accomplished by imposing a small sinusoidal voltage with peak value 0.01 v within a sweeping frequency range from 2 Hz to 6 Hz with the addition of a constant DC bias 0.2 v and the ratio of total applied potential to measured current indicates the total resistance of the electrochemical cell, which is the sum of Rₗₑₐₖ and Rₛₒₗ, as shown in FIG. 16D. The number of accumulated beads increases with time, leading to an increase in total resistance. As a result the dielectric bead concentration is positively correlated to the measured impedance. On the other hand, the impedance change can also be converted to the current change when the applied potential is fixed. This can be realized by SWV, in which the system is perturbed with an applied potential composed of an alternating square wave of a constant amplitude and frequency superimposed by a staircase potential, the current is sampled twice during each square wave cycle, one at the end of the forward pulse, and again at the end of the reverse pulse, and the difference current is plotted versus the potential staircase. Because of the potential window we chose from the previous CV measurement, the Faradaic process is absent as shown in FIG. 16C. It is otherwise visible in SWV and indicated by the peak current. That the current decreases with time is additional evidence of the increased number of accumulated dielectric beads. In order to approximately quantify the concentration of the input dielectric beads, a standard curve of known concentration of beads versus impedance change or current change has to be generated as a reference.

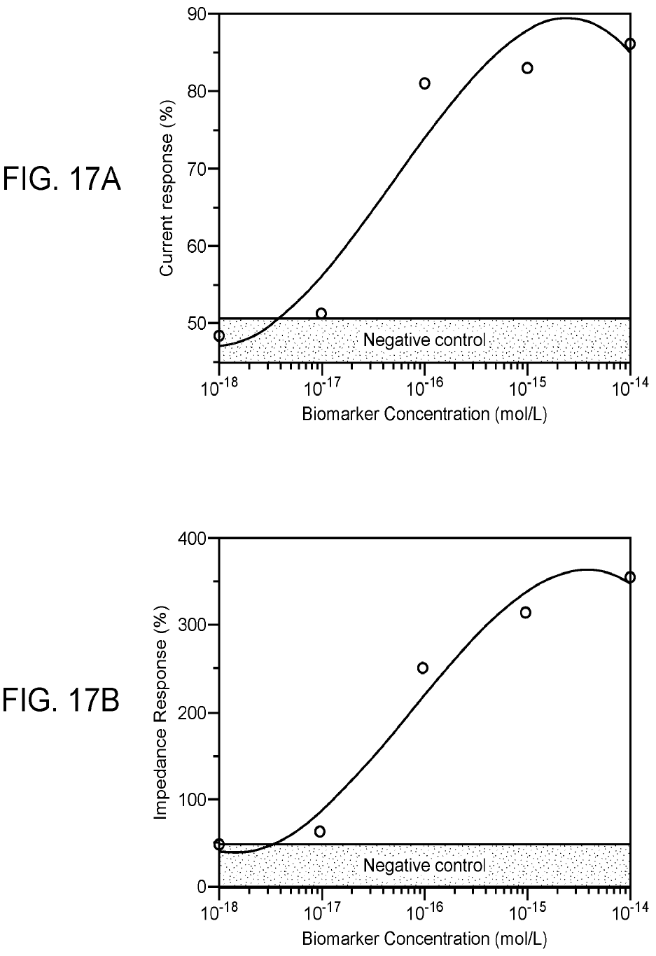

FIGS. 17A and 17B illustrate the relative impedance and current changes for different PSA concentrations after flowing the converted dielectric bead solution for 10 min. A detection limit of 10 aM is observed, and as it is consistently shown by current and impedance response that higher PSA concentration leads to larger current and impedance changes. Here in each flow through experiment corresponding to each PSA concentration, the absolute current and impedance values were extracted from SWV and EIS measurements at 0.3 v applied potential and 4 Hz sweeping frequency, respectively. In order to eliminate the non-uniformity from the nanohole array chips, the current and impedance responses to the B2B converted dielectric beads from each PSA concentration is normalized using the equation:

−ΔG/G₀[%]=[(I₀−I)/I₀]100

ΔG/G₀[%]=[(R−R₀)/R₀]100

I₀ and R₀ are the initial current and impedance values before flowing dielectric beads through nanoholes and I and R are the current and impedance values after flowing through the beads for 10 min. A negative control without PSA was also performed to verify the success of the B2B conversion process and to indicate the limit of detection of PSA.

FIG. 13. (a) B2B conversion scheme converting a protein of interest into sub-micron sized dielectric beads. (b) The quantification of protein of interest is achieved by quantifying the electrochemical response to the accumulation of the converted beads on the surface of nanohole array sensor through cross flow regime.

FIG. 14 Detailed illustration of B2B conversion scheme. (a) magnetic and dielectric beads were pre-functionalized by capture antibodies and detection antibodies, respectively. (b) All the beads are mixed with target solution that may or may not contain a protein of interest. The two bead complex is isolated and purified magnetically if the protein of interest is present. (c) After eluting the two-bead complex, the dielectric beads are disassociated from the magnetic beads (d) The presence of the protein of interest correlates with the elution submicron sized dielectric beads. Scanning electron micrographs of the beads are shown. In some experiments, no converted dielectric beads are observed under SEM when the protein of interest were absent.

FIG. 15 Cyclic voltammetry was used to determine the scanning potential window in order to eliminate the Faradaic reactions occurring on the gold surface. (a) The potential was scanned from −0.3 to 0.5 V, hydrogen evolution reaction and oxidation reaction were observed at different voltages. (b) As the potential window was shrunk to a range from 0.2 to 0.4 V in order to exclude the oxidation reaction, no Faradaic reactions were observed in this approximately rectangular loop.

FIG. 16. (a) An electrochemical cell including a nanohole array with gold coating and filled with phosphate buffered saline was electrically equivalent to a simple circuit composed of a RC circuit in series with a resistor. (b) The dielectric beads were purposely chosen to be larger than the nanoholes to occlude the channels for the ionic movements. An SEM image shows the enriched dielectric beads on the nanohole array. (c-d) SWV and EIS results show the real time current and impedance signals that were changing under flowing dielectric beads through the nanoholes.

FIG. 17. (a-b) Normalized current and impedance responses to the enrichment of the B2B converted dielectric beads under the disclosed cross flow method are shown. The shaded region represents the negative control experimental result when the PSA was absent. A limit of detection is around 10 aM. As PSA concentration increased, more dielectric beads were converted, leading to greater current and impedance signal changes.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1-27. (canceled)

28. A method of determining concentration of an analyte in a solution, the method comprising:
adding to the solution, a plurality of magnetic beads each of which includes a first binding agent that is specific to the analyte;
adding to the solution, a plurality of dielectric nanobeads each of which has a diameter of less than 1,000 nm and includes a second binding agent that is specific to the analyte;
forming a plurality of complexes each of which includes at least one magnetic bead of the plurality of magnetic beads bound to the analyte by the first binding agent and at least one dielectric nanobead of the plurality of dielectric nanobeads bound to the analyte by the second binding agent;
applying a magnetic field to the plurality of complexes to retain the plurality of complexes;
eluting the dielectric nanobeads from the plurality of retained complexes to form a plurality of surrogates;
collecting the surrogates on a surface of a sensor;
determining a number of the surrogates on the surface based on a property of the sensor; and
comparing the number of surrogates on the surface to a standard to determine the concentration of the analyte in the solution as low as 10 aM.

29. The method according to claim 28 where the property of the sensor is selected from the group consisting of a resonance wavelength shift, an optical intensity, an electrical impedance, and an electrical current.

30. The method according to claim 28 where the surface includes a nanohole array including a plurality of nanoholes each of which is smaller than each of the dielectric nanobeads.

31. The method according to claim 30, wherein collecting the surrogates further includes delivering the dielectric nanobeads by microfluidics.

32. The method according to claim 31, wherein collecting further includes flowing the plurality of dielectric nanobeads through the nanohole array.

33. The method according to claim 28, wherein the eluting removes all of the second binding agent from the surrogates.

34. The method according to claim 28, further including generating a standard concentration curve of known concentration of the surrogates.

35. The method according to claim 34, where the standard concentration curve is linear over a range of at least 6 orders of magnitude.

36. The method according to claim 34, wherein a first point in the standard concentration curve is 10 aM.

37. An apparatus for determining concentration of an analyte in a solution, the apparatus comprising:
a fluid circuit for receiving:
a solution including an analyte;
a plurality of magnetic beads each of which includes a first binding agent that is specific to the analyte; and
a plurality of dielectric nanobeads and includes a second binding agent that is specific to the analyte,
wherein the plurality of magnetic beads and the plurality of dielectric nanobeads form a plurality of complexes each of which includes at least one magnetic bead of the plurality of magnetic beads bound to the analyte by the first binding agent and at least one dielectric nanobead of the plurality of dielectric nanobeads bound to the analyte by the second binding agent;
a magnet applying a magnetic field to a portion of the fluid circuit to retain the plurality of complexes;
a first inlet fluidly coupled to the fluid circuit, the first inlet receiving an eluting solution for eluting the dielectric nanobeads from the plurality of retained complexes to form a plurality of surrogates; and
a sensor having a nanohole array for collecting the surrogates on a surface thereof, the sensor outputting a sensor signal corresponding to a concentration of the analyte in the solution as low as 10 aM based on a number of the surrogates on the surface of the sensor.

38. The apparatus according to claim 37, wherein the nanohole array has a plurality of nanoholes each of which is smaller than each of the dielectric nanobeads.

39. The apparatus according to claim 38, wherein the sensor includes an opaque film and each nanohole has a size in a subwavelength of light.

40. The apparatus according to claim 39, wherein the sensor signal is a spectral resonance shift in incident light transmitted through the sensor.

41. The apparatus according to claim 39, wherein the sensor includes a plurality of nanohole arrays each of which has a unique resonance wavelength.

42. The apparatus according to claim 41, wherein the plurality of nanohole arrays is arranged to have a varying spectral profile.

43. The apparatus according to claim 42, wherein the sensor includes a band pass filter having a transmission window overlapping a largest spectral resonance shift of the nanohole array after collecting the surrogates.

44. The apparatus according to claim 42, wherein the concentration of the analyte is encoded on the surface by arranging the nanoholes in an array in a text format.

45. An apparatus for determining concentration of an analyte in a solution, the apparatus comprising:
a fluid circuit for receiving:
a solution including an analyte;
a plurality of magnetic beads each of which includes a first binding agent that is specific to the analyte; and
a plurality of dielectric nanobeads and includes a second binding agent that is specific to the analyte,
wherein the plurality of magnetic beads and the plurality of dielectric nanobeads form a plurality of complexes each of which includes at least one magnetic bead of the plurality of magnetic beads bound to the analyte by the first binding agent and at least one dielectric nanobead of the plurality of dielectric nanobeads bound to the analyte by the second binding agent;
a magnet applying a magnetic field to a portion of the fluid circuit to retain the plurality of complexes;
a first inlet fluidly coupled to the fluid circuit, the first inlet receiving an eluting solution for eluting the dielectric nanobeads from the plurality of retained complexes to form a plurality of surrogates;
a sensor having a nanohole array for collecting the surrogates on a surface thereof; and
an electrochemical cell including a first chamber and a second chamber fluidly coupled to the fluid circuit, wherein the sensor is disposed in the electrochemical cell separating the first and second chambers, and the electrochemical cell outputs a sensor signal corresponding to a concentration of the analyte in the solution as low as 10 aM based on a number of the surrogates on the surface of the sensor.

46. The apparatus according to claim 45, wherein the electrochemical cell measures a change in an electrical property of the solution due to a change in flow caused by occlusion of the nanohole array by the surrogates.

47. The apparatus according to claim 46, wherein the electrochemical cell includes a plurality of electrodes for measuring the change in the electrical property.

48. The apparatus according to claim 47, wherein the sensor further includes a conductive layer facing the first chamber and the nanohole array is facing the second chamber.

49. The apparatus according to claim 48, wherein the plurality of electrodes includes:
a working electrode coupled to the conductive layer;
a reference electrode disposed in the second chamber; and
a counter electrode disposed in the second chamber.

50. The apparatus according to claim 47, wherein measurement is performed using at least one of linear potential sweep voltammetry or impedance spectroscopy.
